(19) United States
(12) Patent Application Publication
(10) Pub. No.: US 2026/0157725 A1
(43) Pub. Date: Jun. 11, 2026

(54) Repeatable Ultrasound using Multi-Array Scanners

(71) Applicant: FUJIFILM SonoSite, Inc., Bothell, WA (US)

(72) Inventors: Jimin Zhang, Bellevue, WA (US); Richard Kelly, Bothell, WA (US); Saeed Aliakbari, Snohomish, WA (US); Daniel Shelton, Prosper, TX (US); Thomas Endres, Sagle, ID (US)

(73) Assignee: FUJIFILM SonoSite, Inc., Bothell, WA (US)

(21) Appl. No.: 18/975,909

(22) Filed: Dec. 10, 2024

(51) Int. Cl. A61B 8/00; A61B 34/32

(52) U.S. Cl. CPC A61B 8/4218; A61B 8/4477; A61B 8/4483; A61B 8/461; A61B 8/483; A61B 8/5246; A61B 8/54; A61B 34/32

(57) ABSTRACT

Systems and methods for repeatable ultrasound using multi-array scanners are disclosed. These techniques include one or more robotic manipulators that couple to one or more multi-array ultrasound scanners to perform ultrasound examinations. The system uses ultrasound data generated by the scanner to generate registration data, which is usable to create movement instructions for the robotic manipulator(s) for controlling movement, positioning, and operation of the scanner, another scanner, or an interventional instrument (e.g., needle). In an example, one robotic manipulator uses a scanner to generate ultrasound data usable to determine positioning and orientation for a second robotic manipulator to insert an interventional instrument or to operate a second scanner. In aspects, the multi-array scanner can generate ultrasound data using a first array, and the ultrasound data is usable to determine where and how to use a second array to generate additional ultrasound data.

### BACKGROUND

Ultrasound systems can generate ultrasound images by transmitting sound waves at frequencies above the audible spectrum (e.g., ultrasound) into a body, receiving echo signals caused by the sound waves reflecting from internal body parts, and converting the echo signals into electrical signals for image generation. Because they are non-invasive and non-ionizing, ultrasound systems are used ubiquitously. In some cases, ultrasound is used throughout a patient's treatment to monitor the patient's progress. For example, the treatment of rheumatoid arthritis usually includes serial ultrasound scanning over multiple examinations that can occur periodically (e.g., every three months). For proper assessment of the progression of rheumatoid arthritis, for example, it is imperative that the images generated during these multiple examinations have essentially identical views. Usually, a clinician (e.g., sonographer, doctor, nurse, trained operator, etc.) holds an ultrasound scanner that includes a transducer array that transmits the ultrasound and receives the reflections. However, operator dependency (among different examinations and/or different operators) when using a handheld ultrasound scanner can prevent the level of reproducibility needed to generate matching image views.

Hence, in some cases, robotic systems are used to hold an ultrasound scanner instead of the ultrasound scanner being held by a clinician. However, for many applications, more than one ultrasound scanner is needed to complete an examination. For instance, a high-frequency ultrasound scanner operating at greater than 20 megahertz (MHz) can be used for shallower depths and/or narrower beam widths, and a low-frequency scanner operating at less than 20 MHz can be used for deeper depths and/or wider beam widths. In another example, a linear array is used for some patient anatomies, while a phased array is used for other anatomies. Hence, the robotic system may need to mount and demount multiple ultrasound scanners during an examination. However, it can be both time-consuming and difficult to mount and demount scanners on a robotic arm automatically, especially in a clinical environment, due to the variations of scanner geometries. Further, registration between the robotic arm and the ultrasound imaging plane is critical to provide reproducible images for serial treatments and also to ensure the correct reconstruction of ultrasound images (e.g., three-dimensional (3D) images). However, the registration usually includes positional and/or orientation errors when ultrasound scanners are mounted and demounted, which can result in inconsistent imaging planes across examinations and poor ultrasound images.

Accordingly, conventional ultrasound systems may not be suitable for treatments that require repeatable ultrasound examinations. Further, the use of these ultrasound systems can result in poor patient care.

### SUMMARY

Systems and methods for repeatable ultrasound using multi-array scanners are disclosed. These techniques include one or more robotic manipulators that couple to one or more multi-array ultrasound scanners to perform ultrasound examinations. The system uses ultrasound data generated by the scanner to generate registration data, which is usable to create movement instructions for the robotic manipulator(s) for controlling movement, positioning, and operation of the scanner, another scanner, or an interventional instrument (e.g., needle, scope, injector, extractor, forceps, cutter, catheter). In an example, one robotic manipulator uses a scanner to generate ultrasound data usable to determine positioning and orientation for a second robotic manipulator to insert an interventional instrument or to operate a second scanner. In aspects, the multi-array scanner can generate ultrasound data using a first array, and the ultrasound data is usable to determine where and how to use a second array to generate additional ultrasound data.

In an example, an ultrasound system is disclosed. The ultrasound system includes one or more ultrasound scanners, one or more robotic manipulators, a processor system, and a position controller. The one or more ultrasound scanners can be configured to generate ultrasound data based on received reflections of ultrasound signals transmitted by the ultrasound scanner at a patient anatomy during a first ultrasound scan. The one or more robotic manipulators can be configured to couple to the one or more ultrasound scanners, the one or more robotic manipulators configured to control positioning, movement, and operation of the one or more ultrasound scanners in accordance with a coordinate system. The processor system can be configured to receive the ultrasound data from the one or more ultrasound scanners, generate registration data based on the ultrasound data, and generate, based on the ultrasound data, scan instructions to configure the one or more ultrasound scanners for an imaging mode for a second ultrasound scan. The position controller can be configured to generate movement instructions for the one or more robotic manipulators based on the registration data. In aspects, the movement instructions are configured to cause the one or more robotic manipulators to move to one or more locations in the coordinate system to enable the one or more ultrasound scanners to perform the second ultrasound scan at the one or more locations in accordance with the imaging mode to generate additional ultrasound data.

In another example, an ultrasound system is disclosed. The ultrasound system can include a multi-array scanner, a first robotic manipulator, a second robotic manipulator, a processor system, and a position controller. The multi-array ultrasound scanner can have at least a first array and a second array. In aspects, the multi-array ultrasound scanner is configured to generate ultrasound data based on received reflections of ultrasound signals transmitted by the multi-array ultrasound scanner at a patient anatomy during a first ultrasound scan. The ultrasound data can include first ultrasound data generated using the first array and second ultrasound data generated using the second array. The first robotic manipulator can be configured to couple to the multi-array ultrasound scanner. In aspects, the first robotic manipulator is configured to control positioning, movement, and operation of the multi-array ultrasound scanner in accordance with a coordinate system. The second robotic manipulator can be configured to couple to an interventional instrument. In aspects, the second robotic manipulator is configured to control positioning, movement, and operation of the interventional instrument in accordance with the coordinate system. The processor system can be configured to receive the ultrasound data from the multi-array ultrasound scanner, generate registration data based on the ultrasound data, and generate operating instructions for the second robotic manipulator to operate the interventional instrument based on the ultrasound data. The position controller can be configured to generate movement instructions for the second robotic manipulator based on the registration data. In aspects, the movement instructions are configured to cause the second robotic manipulator to move to one or more locations and orientations in the coordinate system to enable the operation of the interventional instrument at the one or more locations and orientations in accordance with the operating instructions.

In some aspects, a method for repeatable ultrasound using multi-array scanners is disclosed. The method includes generating first ultrasound data based on received reflections of first ultrasound signals transmitted by a first array of a multi-array ultrasound scanner at a patient anatomy. The method also includes generating registration data based on the first ultrasound data. Further, the method includes generating movement instructions for a robotic manipulator coupled to the multi-array ultrasound scanner based on the registration data. In aspects, the movement instructions are configured to cause the robotic manipulator to move to one or more locations in a coordinate system. In addition, the method includes generating second ultrasound data based on received reflections of second ultrasound signals transmitted by a second array of the multi-array ultrasound scanner at the patient anatomy in accordance with the one or more locations.

### BRIEF DESCRIPTION OF THE DRAWINGS

The appended drawings illustrate examples and are, therefore, exemplary implementations and not considered to be limiting in scope. Throughout the drawings, the same numbers are used to reference like features and components.

FIG. 1 illustrates an ultrasound system in an environment for repeatable ultrasound using multi-array scanners.

FIG. 2 illustrates an example multi-array transducer for repeatable ultrasound using multi-array scanners.

FIG. 3 illustrates example characteristics of a multi-array transducer for repeatable ultrasound using multi-array scanners.

FIG. 4 illustrates an example system for repeatable ultrasound using multi-array scanners.

FIG. 5 illustrates an example system for repeatable ultrasound using multi-array scanners.

FIG. 6A illustrates example robotic manipulators for repeatable ultrasound using multi-array scanners.

FIG. 6B illustrates an example state machine for autonomous movement of robotic manipulators.

FIG. 7 illustrates an example environment for repeatable ultrasound using multi-array scanners.

FIG. 8 represents an example machine-learning architecture used to train a machine-learned model.

FIG. 9 represents an example model using a convolutional neural network to process an input image, which includes representations of objects that can be identified via object recognition, such as people or cars.

FIG. 10 illustrates a block diagram of an example computing device that can perform one or more of the operations described herein, in accordance with some implementations.

FIG. 11 depicts a method for repeatable ultrasound using multi-array scanners, in accordance with one or more implementations.

FIG. 12 depicts a method for repeatable ultrasound using multi-array scanners, in accordance with one or more implementations.

### DETAILED DESCRIPTION

Disclosed herein are systems and methods for repeatable ultrasound using multi-array scanners. Conventional ultrasound systems may not be suitable for treatments that require repeatable ultrasound examinations due to various factors, including human-operator dependency when using a handheld ultrasound scanner or a robotic system required to mount and demount multiple ultrasound scanners during an examination, resulting in inconsistent imaging planes across examinations, as well as poor-quality ultrasound images.

The techniques disclosed herein provide systems and methods that enable ultrasound examples to be repeatable. A robotic manipulator can, for example, couple to a multi-array ultrasound scanner to perform an ultrasound examination. The multi-array ultrasound scanner enables ultrasound data to be generated using different arrays, which can be used for different ultrasound modes (linear, phased, low frequency, high frequency, etc.). In aspects, the ultrasound data generated using a first array can be used by the system to control movement of the robotic manipulator and, hence, the scanner to obtain and generate ultrasound using a second array of the scanner. In one example, the first array is used to generate ultrasound data (and ultrasound image data) that includes bones of a patient and the system can use that ultrasound data of the bones to cause the robotic manipulator to move to particular locations and orientations so that the second array can be used to scan tissue proximate to the bones. In another example, a first robotic manipulator is coupled to a first scanner (e.g., multi-array ultrasound scanner) that generates ultrasound data and, based on the ultrasound data generated by the first scanner, the system controls a second robotic manipulator to operate a second scanner or an interventional instrument (e.g., needle).

In some aspects, the system can compensate for motion of the patient, such as movement of the patient's chest due to the patient breathing. Using ultrasound data generated over one or more breathing cycles, the system can predict the motion of the patient and control the robotic manipulator(s) to anticipate the motion of the patient, such as when using a scanner to further scan the patient or when using an interventional instrument on the patient.

In some implementations, the system can control a robot (e.g., humanoid robot) to act as a smart assistant to a clinician and perform suitable actions during a procedure that uses ultrasound, including automatic and continuous movement in an area (e.g., room). The robot can automatically move to a particular region within a room to assist the clinician, perform a next step in a procedure, or move out of the way. The robot can use a multi-array ultrasound scanner to perform an ultrasound examination on a patient, based on instructions from the clinician. In some aspects, the robot can, automatically and without further instruction from the clinician, repeat the ultrasound examination on one or more additional patients (in a care facility, a triage center, etc.). In one example, the robot can start with a FAST protocol for each of multiple patients and, based on what the robot determines during the FAST protocol, can be trained to follow the FAST protocol with another protocol. These and other examples are disclosed herein in more detail.

### Example Ultrasound System

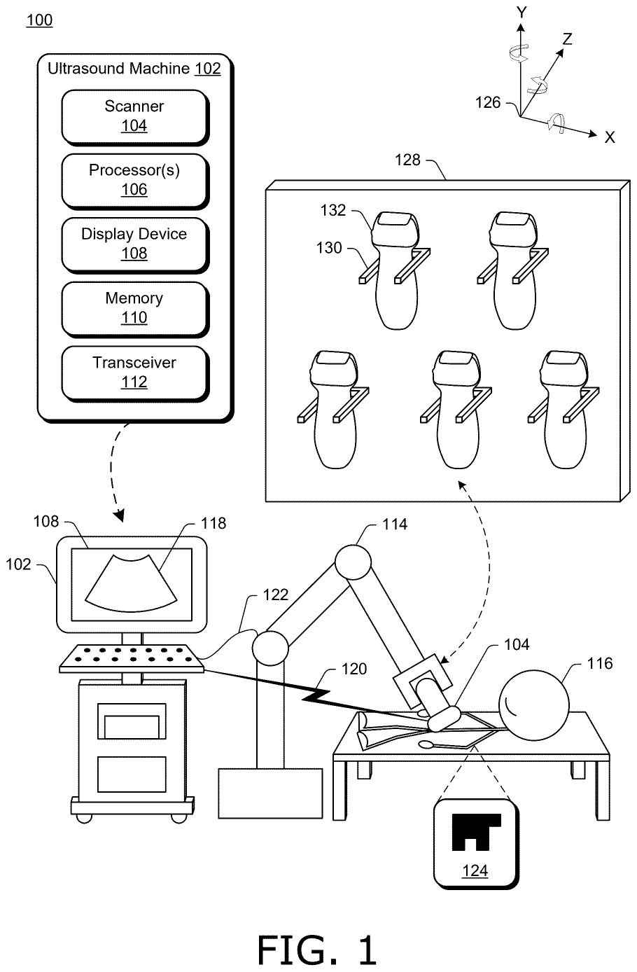

FIG. 1 illustrates an example ultrasound system 100 in an environment for repeatable ultrasound using multi-array scanners during an ultrasound examination in accordance with the disclosed implementations. The ultrasound system in FIG. 1 includes an ultrasound machine 102 and an ultrasound scanner 104. The ultrasound machine 102 generates high-frequency sound waves (e.g., ultrasound) and imaging data based on the ultrasound reflecting off a patient anatomy/body structure and/or an interventional instrument (a needle, a scope, an injector, an extractor, forceps, a cutter, a catheter, etc.). The ultrasound machine 102 includes various components, some of which include the scanner 104, one or more processors 106, a display device 108, a memory 110, and a transceiver 112.

A robotic manipulator 114 (e.g., a robot with one or more robotic arms) holds and directs the scanner 104 toward a patient 116 to non-invasively scan internal bodily structures (e.g., patient anatomies such as organs, tissues, bones, etc.) of the patient 116, an interventional instrument, etc., for testing, diagnostic, therapeutic, or procedural reasons. A robotic manipulator in accordance with the disclosed implementations can include one or more of the robotic manipulators described in U.S. patent application Ser. No. 18/436,699, filed on Feb. 8, 2024, entitled Repeatable Ultrasound to Shelton et al., the disclosure of which is incorporated herein by reference in its entirety.

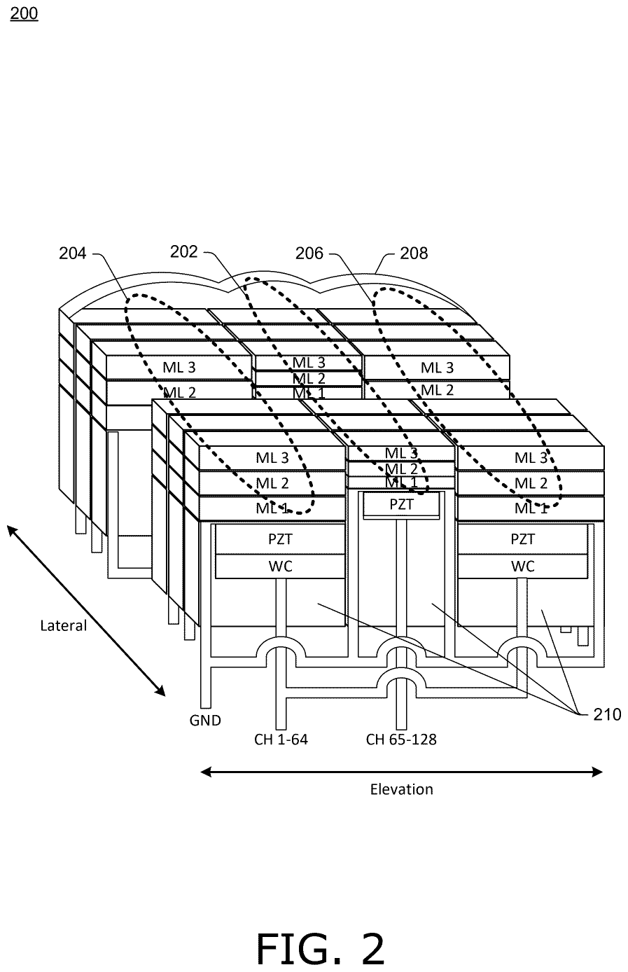

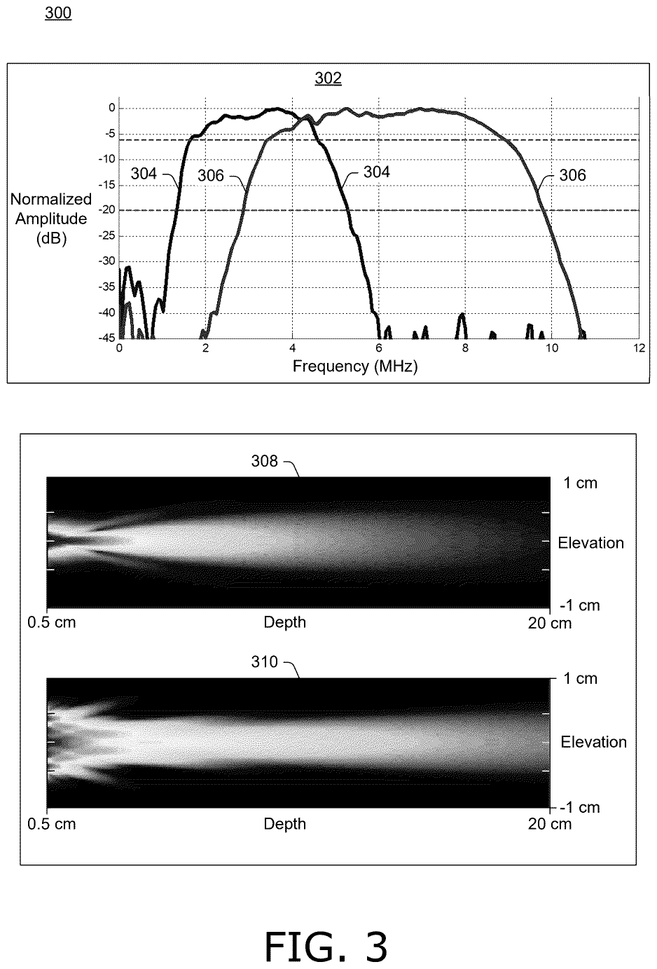

In some implementations, the scanner 104 includes an ultrasound transducer array and electronics communicatively coupled to the ultrasound transducer array to transmit ultrasound signals to the patient anatomy and receive ultrasound signals reflected from the patient anatomy. In some implementations, the scanner 104 is an ultrasound scanner, which can also be referred to as an ultrasound probe or transducer. In implementations, the scanner 104 is a multi-array scanner. For instance, a multi-array scanner in accordance with the present disclosure can include one or more of the arrays described in U.S. patent application Ser. No. 18/613,694, filed on Mar. 22, 2024, entitled Multi-Dimensional and Multi-Frequency Ultrasound Transducers to Zhang et al., the disclosure of which is incorporated herein by reference in its entirety. A multi-array scanner in accordance with the present disclosure can include one or more of the arrays described in U.S. patent application Ser. No. 17/561,313, filed on Dec. 23, 2021, entitled Array Architecture and Interconnection for Transducers to Li et al., the disclosure of which is incorporated herein by reference in its entirety. Further, multi-array scanners for repeatable ultrasound using multi-array scanners are discussed below in more detail with respect to FIGS. 2 and 3.

The display device 108 is coupled to the processor 106, which can include any suitable processor, number of processors, or processor system, such as one or more central processing units (CPUs), graphics processing units (GPUs), vector processors, reduced instruction set computer (RISC) processors, complex instruction set computer (CISC) processors, very long instruction word (VLIW) processors, etc. The processor 106 can execute instructions stored on the memory 110 to perform operations disclosed herein for repeatable ultrasound using multi-array scanners. For example, the processor 106 can process the reflected ultrasound signals to generate ultrasound data, including an ultrasound image (e.g., ultrasound image 118). The display device 108 is configured to generate and display an ultrasound image (e.g., ultrasound image 118) of the anatomy and/or an interventional instrument based on the ultrasound data generated by the processor 106 from the reflected ultrasound signals detected by the scanner 104. In some aspects, the ultrasound data includes the ultrasound image 118 or data representing the ultrasound image 118. The transceiver 112 can be configured to transmit (e.g., over a network maintained by a care facility) the ultrasound data and/or any data related to the ultrasound examination, such as medical worksheet data, etc., to a medical archiver (e.g., a vendor neutral archive (VNA)). In implementations, the transceiver 112 can receive data from the medical archiver, such as patient history data or previous examination data.

The ultrasound scanner 104 is coupled to the ultrasound machine 102 via a coupling 120. In some implementations, the coupling 120 includes a wireless coupling so that the scanner 104 is wirelessly coupled to the ultrasound machine 102 and communicates with the ultrasound machine 102 via one or more wireless transmitters, receivers, or transceivers over a wireless connection or network (e.g., Bluetooth™, Wi-Fi™, etc.). Additionally or alternatively, the coupling 120 can include one or more cables to connect the ultrasound scanner 104 to the ultrasound machine 102.

In implementations, the robotic manipulator 114 is coupled to the ultrasound machine 102 via a coupling 122. The coupling 122 can include a wireless coupling so that the robotic manipulator 114 is wirelessly coupled to the ultrasound machine 102 and communicates with the ultrasound machine 102 via one or more wireless transmitters, receivers, or transceivers over a wireless connection or network (e.g., Bluetooth™, Wi-Fi™, etc.). Additionally or alternatively, the coupling 122 can include one or more cables to connect the robotic manipulator 114 to the ultrasound machine 102. In implementations, the scanner 104 is electronically coupled to the robotic manipulator 114, and the ultrasound machine 102 is in communication with the ultrasound scanner 104 via the coupling 122. For instance, the ultrasound machine 102 can provide transmit waveforms (or definitions thereof) to generate ultrasound to the ultrasound scanner 104 over the coupling 122, and/or the ultrasound scanner 104 can provide ultrasound data to the ultrasound machine 102 over the coupling 122. In some implementations, the robotic manipulator 114 provides a charging signal (e.g., current, voltage, energy, etc.) to the ultrasound scanner 104 to charge a battery of the ultrasound scanner 104. The charging signal can be implemented via a non-contact charger (e.g., inductive charging, radio frequency (RF) or resonance charging, optical charging, etc.) that includes a transmitter on the robotic manipulator 114 and a receiver on the ultrasound scanner 104. In implementations, the robotic manipulator 114 can charge a battery of the ultrasound scanner 104 while the ultrasound scanner 104 is in operation (e.g., when the ultrasound scanner 104 is generating and receiving ultrasound signals). Hence, the ultrasound scanner 104 can be constantly charged and ready for use.

In implementations, the patient 116 is fitted with one or more fiducial markers 124. For instance, the fiducial marker 124 can include a tattoo (e.g., a temporary or permanent ink marking), a sonolucent sticker, and the like. The ultrasound system 100 can use the fiducial marker 124 to register the robotic manipulator 114 and/or the ultrasound scanner 104 to the patient 116. Registration can include determining positional and orientation data according to, for example, a coordinate system 126. In implementations, the registration system includes one or more cameras in the environment to determine the registration data, including relative locations and orientations of the robotic manipulator 114 and/or the ultrasound scanner 104 (e.g., relative to the patient 116).

The ultrasound system in FIG. 1 also includes a panel 128 (e.g., shelf, table, storage compartment, locker, etc.) that includes holders 130, which store available ultrasound scanners 132 that can be retrieved and mounted to the robotic manipulator 114. The robotic manipulator 114 can demount the ultrasound scanner 104 currently held by an arm of the robotic manipulator 114 and place it on one of the holders 130. The robotic manipulator 114 can then select one of the ultrasound scanners 132 from the panel 128 and mount the selected scanner to the arm of the robotic manipulator 114. In implementations, one or more of the ultrasound scanners 132 is a multi-array scanner and the robotic manipulator 114 can perform a procedure (e.g., an ultrasound examination) with a single one of the ultrasound scanners 132, so that the procedure can be performed without the robotic manipulator 114 being required to demount the ultrasound scanner and mount another ultrasound scanner.

Additionally or alternatively, one or more of the robotic manipulator 114, the ultrasound scanner 104, and the patient 116 can be fitted with an inertial measurement unit (IMU) that can be used to determine the positional and/or orientation data in the coordinate system 126. The IMU can include a combination of accelerometers, gyroscopes, and magnetometers usable to generate positional data including, for example, data representing six degrees of freedom (6DOF), such as yaw, pitch, and roll angles in the coordinate system 126. Typically, 6DOF refers to the freedom of movement of a body in three-dimensional space. For example, the body is free to change position as forward/backward (surge), up/down (heave), and left/right (sway) translations in three perpendicular axes, combined with changes in orientation through rotation about three perpendicular axes, often termed yaw (normal axis), pitch (transverse axis), and roll (longitudinal axis). Additionally or alternatively, the ultrasound system 100 can include a camera and one or more fiducial markers on the scanner 104 (not shown in FIG. 1) to determine the positional and/or orientation data for the ultrasound scanner 104.

### Example Ultrasound System

An ultrasound scanner, such as the ultrasound scanner 104, for repeatable ultrasound using multi-array scanners in accordance with the disclosed techniques can include a multi-array scanner (e.g., a multi-array transducer). A multi-array scanner in accordance with the disclosed techniques can include multi-array transducer assemblies having any combination of piezoelectric micromachined ultrasonic transducer (PMUT) array elements, lead zirconate titanate (PZT) array elements, and capacitive micromachined ultrasonic transducer (CMUT) array elements. In implementations, a multi-array scanner for repeatable ultrasound using multi-array scanners can include a first array with array elements selected from the group consisting of PZT, PMUT, and CMUT array elements, as well as a second array with additional array elements selected from the group consisting of PZT, PMUT, and CMUT array elements. The elements of the first array can be of a different type than the elements of the second array (e.g., the first array can include PMUT or PZT elements, and the second array can include CMUT elements). Alternatively, the elements of the first array can be of a same type as the elements of the second array (e.g., the first array and the second array can include PZT elements). The PMUT, CMUT, and/or PZT array elements can be tuned differently to enhance the performances (e.g., by using different tuning inductors or complex impedances).

FIG. 2 illustrates an example multi-array transducer 200 for repeatable ultrasound using multi-array scanners. The multi-array transducer 200 can be included in a multi-array scanner and includes three arrays or sub-arrays, such as a first array 202, a second array 204, and a third array 206. The first array 202 can be referred to as a center array, as it is located between the second array 204 and the third array 206, which can be referred to as adjacent arrays. In the example multi-array transducer 200, the arrays 202, 204, and 206 are laid out in rows, parallel to one another. However, multi-array transducers in accordance with the disclosed implementations are not so limited and can be arranged in any suitable configuration. For example, array configurations can include a circular array configuration (e.g., arrays arranged in concentric circles or ellipses), a polygonal array configuration (e.g., arrays arranged in concentric polygons, such as nested triangles), an open-shaped array configuration (e.g., nested “L” or “V” shaped arrays), and a matrix array configuration (e.g., a configuration that includes a center array with elements on a grid, and a surrounding array that includes array elements that are also on the grid and that surround the center array).

The example multi-array transducer 200 also includes an acoustic lens (e.g., lens 208) that covers the three arrays 202, 204, and 206. In the example in FIG. 2, the lens 208 includes multiple radii of curvature. For example, a first radius covers the first array 202, a second radius covers the second array 204, and a third radius covers the third array 206. In an example, the second radius and the third radius are the same radius, which is different from the first radius. In other implementations, the lens 208 can include a single radius of curvature that covers the three arrays 202-206.

In the example in FIG. 2, the arrays 202, 204, and 206 of the multi-array transducer 200 include array elements of lead zirconate titanate (PZT) ceramic material with piezoelectric properties and acoustic matching layers (ML 1, ML 2, ML 3, etc.). However, multi-array transducers in accordance with the present disclosure are not so limited and can include arrays in any suitable combination of PZT, PMUT, and CMUT array elements. In one example, the center array (e.g., the first array 202) can include PZT array elements, and the adjacent arrays (e.g., the second array 204 and the third array 206) can include PMUT array elements. In another example, the center array can include PZT array elements, and the adjacent arrays can include CMUT array elements. In another example, the center array can include PMUT array elements, and the adjacent arrays can include CMUT array elements. In another example, the center array can include PMUT array elements, and the adjacent arrays can include PZT array elements. In another example, the center array can include CMUT array elements, and the adjacent arrays can include PMUT array elements. In another example, the center array can include CMUT array elements, and the adjacent arrays can include PZT array elements. In aspects, the array elements are stacked between a backing material 210 and the lens 208.

In implementations, the first array 202 operates at a first frequency, and the second and third arrays 204 and 206, respectively, operate at a second frequency that is different from the first frequency. For instance, the second frequency can be lower than the first frequency. Alternatively, the second frequency can be higher than the first frequency. In some implementations, the second and third arrays 204 and 206, respectively, operate at different frequencies from one another, which can be higher or lower than the first frequency. The frequencies of the arrays 202, 204, and 206 can be selected such that the bandwidths of the arrays 202-206 overlap and such that the union of the individual bandwidths of the arrays 202, 204, and 206 extends the overall bandwidth of the multi-array transducer 200.

For example, FIG. 3 illustrates example characteristics 300 of a multi-array transducer usable for repeatable ultrasound using multi-array scanners. The characteristics 300 include a frequency response 302 of a multi-array transducer, such as the multi-array transducer 200 in FIG. 2. The frequency response 302 includes a first bandwidth 304 and a second bandwidth 306. The first bandwidth 304 illustrates a frequency response of an array, such as the arrays 204 and 206 in FIG. 2, and the second bandwidth 306 illustrates a frequency response of another array, such as the array 202 in FIG. 2. By combining the first bandwidth 304 and the second bandwidth 306, the overall bandwidth of the multi-array transducer is increased, allowing the system to be configured in various operation modes. Example operation modes are described in Table 1.

Table 1 illustrates operation modes and transducer-array configurations for a three-array transducer array, such as the example illustrated in FIG. 2. In Table 1, the center array represents the array 202, a first adjacent array represents the array 204, and a second adjacent array represents the array 206. Also, “LF” represents low frequency, “HF” represents high frequency, “Tx” refers to transmission, “Rx” refers to reception, and “THI” refers to tissue harmonic imaging.

TABLE 1
Example operation modes and transducer array configurations
for a three-array transducer array

| | Near Field | | | Far Field | | |
|---|---|---|---|---|---|---|
| | First | | Second | First | | Second |
| | Adjacent | Center | Adjacent | Adjacent | Center | Adjacent |
| Operation Mode | Array | Array | Array | Array | Array | Array |
| Mode 1 | Not Used | Linear | Not Used | Phased | Not | Phased |
| (Linear and | | HF | | LF | Used | LF |
| Phased) | | (Tx/Rx) | | (Tx/Rx) | | (Tx/Rx) |
| Mode 2 | LF | Linear | LF | Phased | Linear | Phased |
| (Transmitting/ | (Tx) | HF | (Tx) | LF | HF | LF |
| Receiving | | (Rx) | | (Tx) | (Rx) | (Tx) |
| imaging, | | | | | | |
| Broadband THI | | | | | | |
| with separated | | | | | | |
| aperture) | | | | | | |
| Mode 3 | LF | Linear | LF | Phased | Linear | Phased |
| (Broadband THI | (Tx) | HF | (Tx) | LF | HF | LF |
| with overlap | | (Tx/Rx) | | (Tx) | (Tx/Rx) | (Tx) |
| aperture) | | | | | | |
| Mode 4 | LF | Linear | LF | Phased | Linear | Phased |
| (Full Aperture and | (Tx/Rx) | HF | (Tx/Rx) | LF | HF | LF |
| Broadband THI) | | (Tx/Rx) | | (Tx/Rx) | (Tx/Rx) | (Tx/Rx) |
| Mode 5 | LF | Linear | LF | Phased | Linear | Phased |
| (Transmitting/ | (Rx) | HF | (Rx) | LF | HF | LF |
| Receiving imaging | | (Tx) | | (Rx) | (Tx) | (Rx) |
| for Sub-harmonic | | | | | | |
| imaging with | | | | | | |
| separated | | | | | | |
| aperture) | | | | | | |

The characteristics 300 also include illustrations of a first ultrasound beam 308 and a second ultrasound beam 310 showing depth against elevation. The first ultrasound beam 308 corresponds to the first array 202 in FIG. 2 and the second ultrasound beam 310 corresponds to the second and third arrays 204 and 206, respectively, in FIG. 2. In this example, because the first array 202 is implemented to operate at a higher frequency than the second and third arrays 204 and 206, the second ultrasound beam 310 has deeper penetration than the first ultrasound beam 308, but the first ultrasound beam 308 has better focus than the second ultrasound beam 310. Hence, the multi-array transducer can exploit the different ultrasound beam profiles associated with the multiple arrays to image at multiple depths with the same ultrasound scanner, rather than requiring the use of multiple ultrasound scanners. Thus, a robotic manipulator, such as the robotic manipulator 114, can perform a full-body ultrasound scan of a patient with a single scanner, without the need of mounting and demounting multiple scanners, thus saving time and resources, reducing the chances of infection due to scanner change, and maintaining registration of the robotic arm to the imaging plane.

Moreover, because the transducer can include multiple arrays of different types of array elements (e.g., PZT, PMUT, and CMUT), the strengths of each of the types of array elements can be exploited. For example, PMUT, which conventionally has better transmit sensitivity than CMUT, can be used for ultrasound transmission, while CMUT, which conventionally has better receive sensitivity than PMUT, can be used for ultrasound reception.

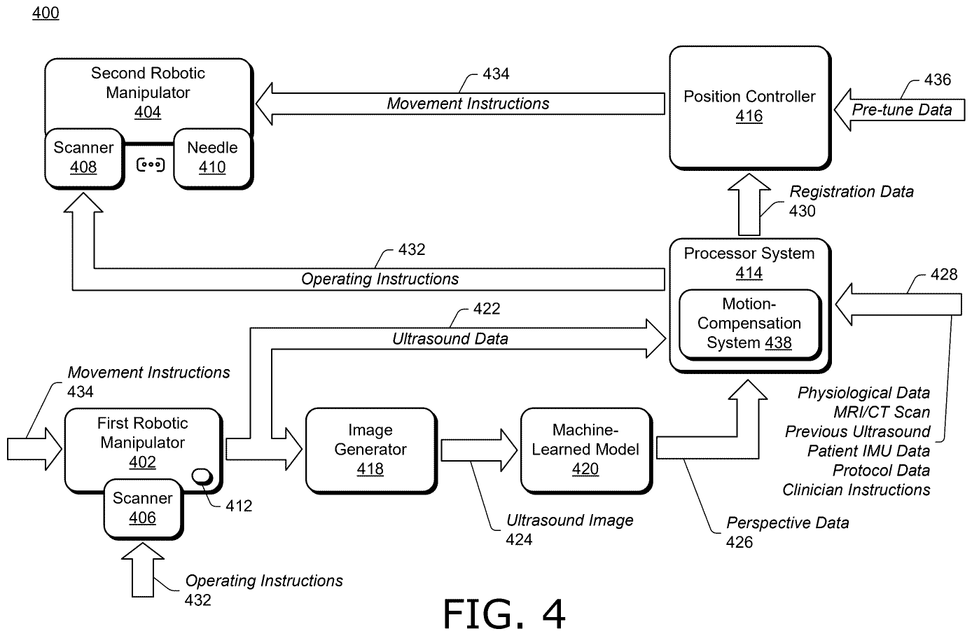

FIG. 4 illustrates an example system 400 for repeatable ultrasound using multi-array scanners. The system 400 includes a first robotic manipulator 402 and a second robotic manipulator 404. The first robotic manipulator 402 is configured to hold an ultrasound scanner (e.g., a first scanner 406), and the second robotic manipulator 404 is configured to hold a medical device, including an ultrasound scanner (e.g., a second scanner 408), an interventional instrument (e.g., a needle 410), or any other suitable medical device. In implementations, the first robotic manipulator 402 includes a first arm configured to hold the first scanner 406, and the second robotic manipulator 404 includes a second arm configured to hold the second scanner 408 or the needle 410. The first arm and the second arm can be implemented on a same robot or on different robots.

In implementations, the first scanner 406 on the first robotic manipulator 402 acts as a scout scanner that can obtain information (e.g., ultrasound data), which the system 400 can use to control the second robotic manipulator 404, the second scanner 408, and/or the needle 410. For instance, a robot can move the first scanner 406 with the first robotic manipulator 402 along a patient and follow the first robotic manipulator 402 with the second robotic manipulator 404, whose movement is based on data captured by the first scanner 406. For example, based on a bone imaged by the first scanner 406, the system 400 can provide movement instructions to the second robotic manipulator 404 and operating instructions (e.g., scan instructions) to the second scanner 408 to image tissue near the bone. In another example, based on ultrasound image data captured by the first scanner 406 that depicts a blood vessel, the system can provide (i) movement instructions to the second robotic manipulator 404 to move to a position proximate to the blood vessel and (ii) instructions to insert the needle 410 into the blood vessel.

The first robotic manipulator 402 includes one or more sensors 412. Not shown for clarity, the second robotic manipulator 404 can also include one or more sensors like the sensors 412. The one or more sensors 412 can include any suitable type of sensor, including a light sensor (a laser, light detection and ranging (LIDAR), etc.), a pressure sensor, a camera, a line scanner, an ultrasound array, etc., and the system 400 can use sensor data obtained by the one or more sensors 412 to generate registration data. For instance, the one or more sensors 412 can read a fiducial marker (e.g., the fiducial marker 124 in FIG. 1) to generate the registration data.

The system 400 also includes a processor system 414, a position controller 416, an image generator 418, and a machine-learned model 420. Ultrasound data 422 (analog-to-digital convert (ADC) samples, beamformed samples, non-scan converted data, scan-converted data, etc.), obtained by the first scanner 406 that is mounted to the first robotic manipulator 402, is provided to the image generator 418 and the processor system 414. The image generator 418 generates one or more ultrasound images 424 from the ultrasound data 422 and provides the ultrasound images 424 to the machine-learned model 420. Although not illustrated in FIG. 4 for clarity, the image generator 418 can additionally or alternatively provide the ultrasound images 424 to the processor system 414. In implementations, the sensor data from the sensors 412 is grouped with the ultrasound data 422 and provided along with the ultrasound data 422 to the processor system 414 and/or the image generator 418.

The machine-learned model 420 receives the ultrasound images 424 from the image generator 418 and generates perspective data 426 that can represent a perspective for the ultrasound image 424. For example, the perspective data 426 can include a vector that represents an angle of a viewer or source of the ultrasound image 424. The vector can be relative to a coordinate system. In an example, the vector can be relative to a previous ultrasound image. In implementations, the perspective data 426 includes data representing an image view or view of anatomy in an ultrasound image. In implementations, the perspective data 426 includes an amount of movement of a perspective from one ultrasound image to another ultrasound image. The movement can be caused by a patient's breathing, movement of a transport vehicle (e.g., an ambulance, life flight) transporting the patient, movement of an ultrasound scanner, and the like. The machine-learned model 420 provides the perspective data 426 to the processor system 414.

The processor system 414 receives the ultrasound data 422 from the first scanner 406 mounted on the first robotic manipulator 402, the perspective data 426 from the machine-learned model 420, and various secondary data 428, such as physiological data, magnetic resonance imaging (MRI) scan data or computed tomography (CT) scan data, previous ultrasound data, patient IMU data, protocol data, clinician instructions, and the like. The physiological data can include any suitable data representing a physiological process of the patient, such as electrocardiogram (ECG) data, breathing/respiratory data, etc. The MRI/CT scan data can include imaging data for the patient other than ultrasound data, such as imaging data from an MRI scan, CT scan, etc. The previous ultrasound data can include ultrasound data (e.g., ultrasound images) from one or more previous ultrasound examinations for the patient. The patient IMU data can include positional and orientation data from one or more IMUs worn by the patient. The protocol data can include any configuration data for the system pertaining to an ultrasound protocol, such as a current protocol step, a next protocol step, instructions for configuring an ultrasound scanner and/or ultrasound machine for performing a protocol step, etc. The clinician instructions can include user input from a clinician for the system 400, such as configuration data for the system, movement instructions for one or more of the robotic manipulators, etc.

Based on the data received by the processor system 414, the processor system 414 generates registration data 430 for one or more components of the system 400. The registration data 430 can include positional and/or orientation data for any component of the system 400, including the first robotic manipulator 402, the second robotic manipulator 404, the first scanner 406, the second scanner 408, the needle 410, the sensors 412, the patient, etc. The positional and/or orientation data can represent these components in a coordinate system (e.g., 6DOF in the coordinate system 126). The processor system 414 provides the registration data 430 to the position controller 416. The processor system 414 also generates operating instructions 432 for the first scanner 406 and/or the second scanner 408. The operating instructions 432 can configure the scanners for an imaging mode (B-mode, super harmonic tissue imaging, sub-harmonic tissue imaging, etc.) and instruct the scanner when to scan (e.g., at what positions in the coordinate system to scan). In some aspects, the operating instructions 432 can be provided to the second robotic manipulator 404 for operating a medical device such as the needle 410. Example operating instructions for operating the needle 410 include inserting the needle 410 into the patient to a predefined depth, pushing a plunger of the needle 410 to inject a fluid into the patient, pulling the plunger of the needle 410 to withdraw fluid from the patient, etc.

The position controller 416 receives the registration data 430 from the processor system 414 and generates movement instructions 434 for the robotic manipulators 402 and 404. The movement instructions 434 can instruct the robotic manipulators 402 and 404 to move to a location in the coordinate system, including an orientation (e.g., 6DOF). For instance, a robotic manipulator can implement one or more axes of operation (e.g., a robotic manipulator can include multiple joints that couple components of the robotic manipulator that can operate independently with 6DOF). The movement instructions 434 can instruct each of the components in each of the axes of operation to move to a particular location with a particular orientation. In this way, a robotic manipulator can place a device mounted to it (e.g., an ultrasound scanner or interventional instrument) at any desired location and with any desired orientation in the coordinate system. For example, the movement instructions 434 can instruct the second robotic manipulator 404 to move to a particular location with a particular orientation and insert the needle 410 into the patient to a predefined depth determined based on the registration data 430. In another example, the movement instructions 434 can instruct the first robotic manipulator 402 to move to a particular location with a particular orientation for additional scanning of the patient with the scanner 406.

In implementations, the movement instructions 434 are based on pre-tune data 436 received by the position controller 416. The pre-tune data 436 can include any suitable data to instruct a robotic manipulator to move that may not be based on data generated by the ultrasound scanners or that is not part of the secondary data 428 received by the processor system 414. For instance, the pre-tune data 436 can include initial movement instructions (e.g., positional and/or orientation data) for a robotic manipulator from a previous ultrasound examination, default values for positional and/or orientation data, etc. By using the pre-tune data 436, the system 400 can begin scanning automatically, without explicit positional and/or orientation instructions provided by a clinician or derived from a current use of the system 400.

In an example, the pre-tune data 436 is based on reading a fiducial marker (e.g., the fiducial marker 124). For instance, the fiducial marker can include a quick-response (QR) code placed on the patient by a clinician. The QR code can include data to instruct the system 400 to use a robotic manipulator and multi-array scanner, data to instruct the system 400 on particular ultrasound images to capture, a protocol to follow, etc. Hence, a clinician can perform a first ultrasound examination and convey instructions to a robotic manipulator via the QR code so that the robotic manipulator can repeat the examination, even in the absence of the clinician.

In implementations, the system 400 implements a motion-compensation system 438 that can position and orient a robotic manipulator (e.g., the second robotic manipulator 404) to dynamically adjust for a motion. For instance, the motion can be due to a patient's movement, such as due to breathing. Hence, the system 400 can include a respiratory sensor to indicate a patient's breathing cycle. Additionally or alternatively, the first scanner 406 can generate data for multiple ultrasound images over one or more cycles of the patient's breathing cycle to create a history of images (e.g., a set of ultrasound images). For example, the motion-compensation system 438 can determine positional differences of the patient anatomy over a set of ultrasound images generated over a duration of time, such as one or more breathing cycles of the patient. Using this history of images, the processor system 414 can predict a next position of the patient anatomy relative to a current position and include an offset in the registration data 430 representing this prediction. The position controller 416 can then generate the movement instructions 434 for the second robotic manipulator 404 so that ultrasound data captured by the second scanner 408 is from a position to compensate for the motion. In this way, the system 400 can generate consistent and stable ultrasound images that reduce artifacts caused by motion. By reducing the artifacts caused by motion, a clinician can, for example, during a needle procedure, be confident that a needle tip is being inserted at a desired location and at a time of the breathing cycle that is not affecting the ultrasound images generated by the system 400. Other sources of motion for which the system 400 can compensate in this way include motion caused by transport, such as in a helicopter or ambulance.

In implementations, the system 400 includes one or more multi-array ultrasound scanners, such as the first and second scanners 406 and 408. By using a multi-array ultrasound scanner, the system 400 can image at both shallow and deep tissue depths and in multiple operation modes that are simply not possible with the use of conventional ultrasound scanners. Thus, the system 400 can perform a full-body scan without demounting an ultrasound scanner and mounting an additional ultrasound scanner. In an example, both the first scanner 406 and the second scanner 408 are implemented as multi-array scanners, and the system 400 implements a calibration routine to determine positional and orientation data to align the imaging planes of the first and second scanners 406 and 408. The system 400 can then perform an examination (e.g., a full-body scan) on a patient by simultaneous operation of the first and second scanners 406 and 408, reducing the time of the examination data. In one example, the system 400 can compare the results generated by the first and second scanners 406 and 408 for a same anatomy and generate a confidence score based on the comparison. For instance, if the imaging results from the two scanners differ by more than a threshold value, then the system 400 can generate a failing score. Otherwise, the system 400 can generate a passing score. The system 400 can implement one or more machine-learned models for the comparison. For instance, the machine-learned model can simultaneously process ultrasound images from both the first scanner 406 and the second scanner 408 and generate the confidence score.

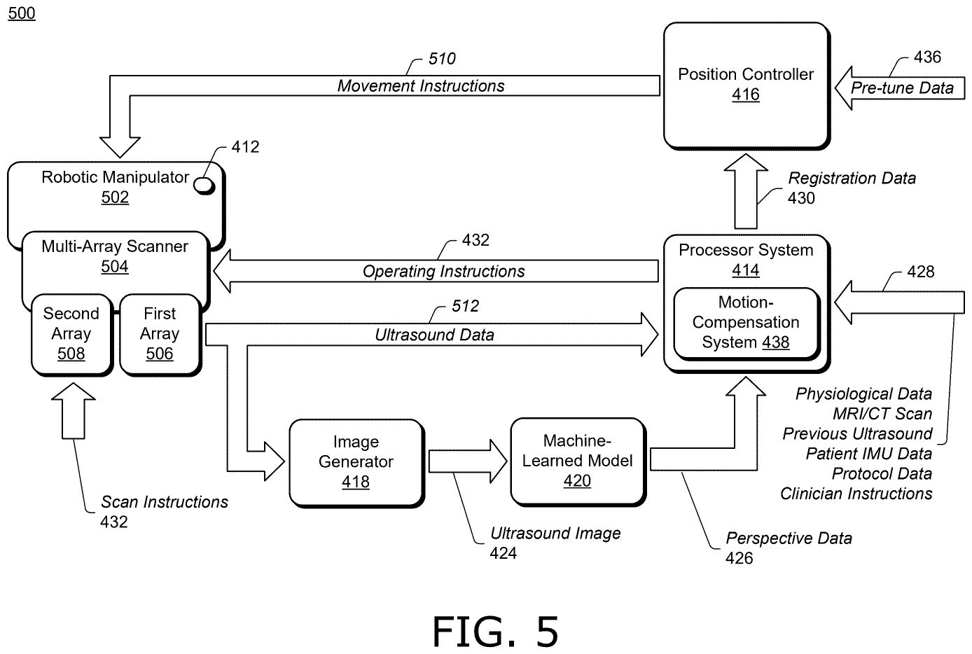

FIG. 5 illustrates an example system 500 for repeatable ultrasound using multi-array scanners. The system 500 includes some of the same components as the system 400 in FIG. 4, but rather than including the two robotic manipulators (e.g., the first robotic manipulator 402 and the second robotic manipulator 404), the system 500 includes a single robotic manipulator 502 (e.g., a single robot arm with multiple joints and axes) that mounts a multi-array scanner 504. The multi-array scanner 504 includes a first array 506 and a second array 508. The array 202 in FIG. 2 is an example of the first array 506, and the second and third arrays 204 and 206 in FIG. 2 are examples of the second array 508.

In implementations, the first array 506 can be used as a scout array, in the sense that based on ultrasound data generated by the first array 506, the system 500 can generate movement instructions 510 for the robotic manipulator 502 and scan instructions (e.g., the operating instructions 432) for the multi-array scanner 504 so that the second array 508 is used to generate ultrasound data in a desired image plane and with a desired view/perspective. In this way, the robotic manipulator 502 has no need to switch scanners (e.g., by decoupling the multi-array scanner 504 and coupling to a different scanner). Using the same scanner enables different scanning modes (e.g., linear, phased) to be used on a patient anatomy based on the same registration data 430.

Additionally or alternatively, the system 500 can implement motion compensation by generating an offset in location and/or orientation based on a history of ultrasound images obtained via the first array 506. For instance, the history of ultrasound images can span one or more breathing cycles, and based on the motion caused by the breathing cycles, the system 500 can generate the movement instructions 510 for the robotic manipulator 502 according to the offset in location and/or orientation. Based on the movement instructions 510, the robotic manipulator 502 can orient the multi-array scanner 504 so that ultrasound images captured via the second array 508 are stable and without artifacts caused by the motion.

In implementations, the first array 506 generates image data that includes bones of the patient, and the system 500 generates movement instructions 510 for the robotic manipulator 502 to move to one or more locations and orientations in a coordinate system so that the second array 508 can generate ultrasound data 512 that includes tissue proximate to the bones. The system 500 can display one or more images (not shown for clarity in FIG. 5) that depict the bones as a map/trajectory with a current location on or near the bones, and an ultrasound image of the soft tissue at or near the location.

In an example, the first array 506 and the second array 508 can image a patient anatomy using different imaging planes, and the system 500 can generate a 3D image of the patient anatomy based on the ultrasound data 512 from the first array 506 and the second array 508. Additionally or alternatively, the system 500 can generate a full-body scan of a patient using the first array 506 and the second array 508 of the multi-array scanner 504 and not using any other array of another ultrasound scanner. A full-body scan can include one or more protocols, such as a Focused Assessment with Sonography for Trauma (FAST) protocol, a Rapid Ultrasound for Shock and Hypotension (RUSH) protocol, a Venous Excess Ultrasound (VExUS) protocol, and the like. Additionally or alternatively, a full-body scan can include a lung exam, a right upper quadrant exam, a left upper quadrant exam, a cardiac exam, a pelvic exam, etc.

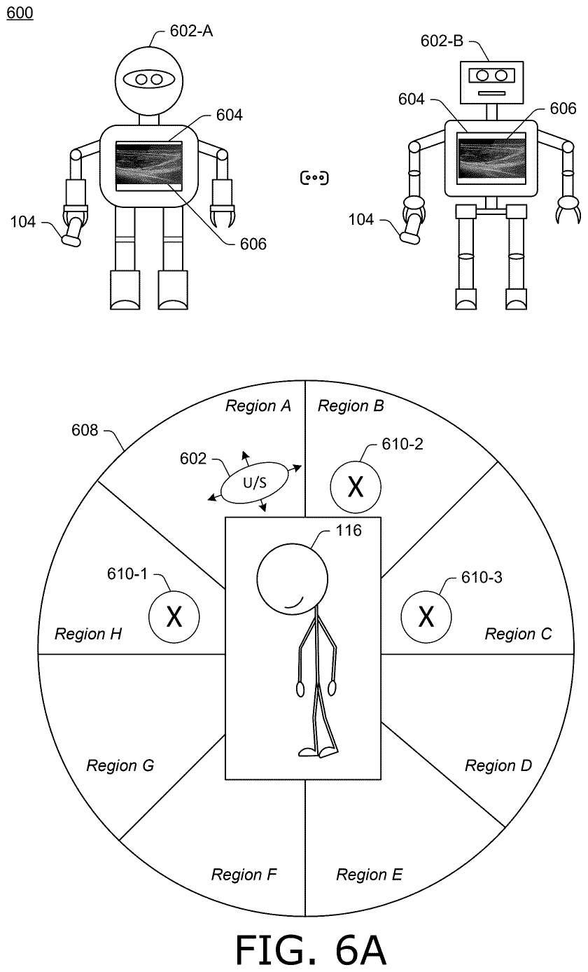

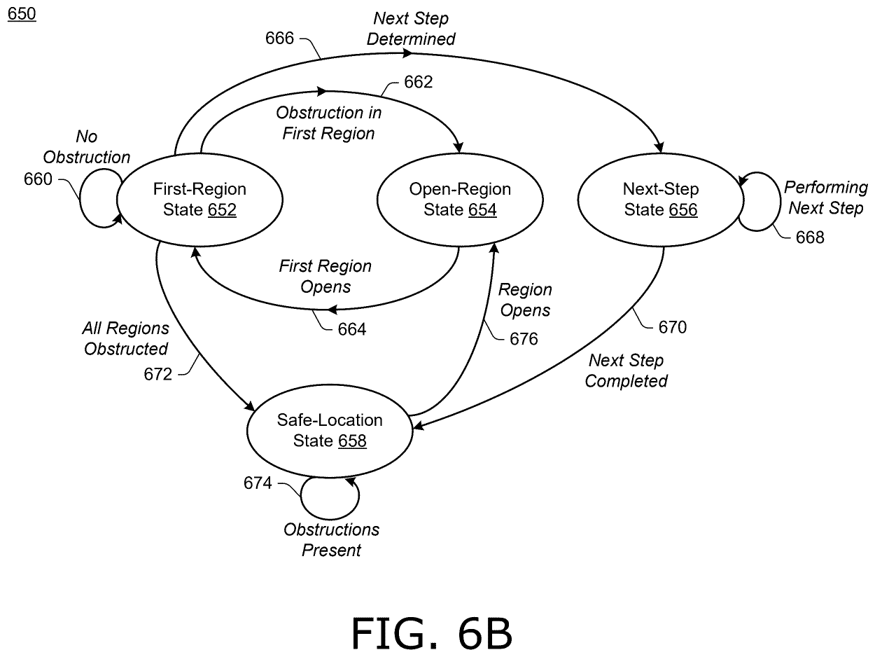

FIG. 6A illustrates example robotic manipulators 600 for repeatable ultrasound using multi-array scanners. The robotic manipulators 600 include a first robot 602-A and a second robot 602-B (collectively robots 602). The robots 602 are humanoid robots in that they have human form factors. The first robot 602-A and the second robot 602-B are examples of the first robotic manipulator 402 and the second robotic manipulator 404, respectively, from FIG. 4. In this way, the first robotic manipulator 402 and the second robotic manipulator 404 are separate robots. In another example, the first robotic manipulator 402 and the second robotic manipulator 404 are coupled to the same robot, such as two arms of the first robot 602-A. In aspects, the robots 602-1 and 602-2 can hold (e.g., couple to) and operate one or more ultrasound scanners, examples of which include the ultrasound scanner 104, the first scanner 406, the second scanner 408, the multi-array scanner 504, and the like. The robots 602 can include a display device 604 on their torso, for example. The display device 604 can be configured to display a user interface that can display, for example, ultrasound images 606.

The robots 602 can act as smart assistants to a clinician and can perform any suitable action during a procedure that uses ultrasound, including automatic and continuous movement, examples of which include turning and orienting a screen (e.g., the display device 604) toward an operator, moving to a desired distance or location from the operator, movement based on the operator being right-handed or left-handed, adjusting a height, sensing a position of other people in the room, retreating to a safe space or position if a patient is crashing, and moving a probe to scan a particular location on the patient, and so on. In aspects, the movement is predictive such that the robot 602 anticipates a next step to enable the robot 602 to be positioned in a correct location for the next step or to be located out of the way of the operator. In some implementations, the movement is region-based, such as according to regions 608 illustrated in FIG. 6A.

During a procedure that uses ultrasound, the robots 602 can act as participatory assistants to the clinician. Example actions that the robots 602 can perform include retrieving a medical instrument for the operator, adjusting environmental controls (heating, ventilation, and air conditioning (HVAC), lighting, temperature, fan, etc.) of the room, retrieving and/or dispensing medication, calling for a second operator, recording and/or transcribing dictation, recording events, providing audible count-down, and providing voice-output describing time remaining or other alerts. Some additional actions can include operating equipment (e.g., vacuum pump), automatically operating one of multiple probes based on a determination of a protocol step, wiping sweat from a surgeon, etc. The robots 602 can include an advanced user interface, such as for performing a neural link procedure.

The regions 608 illustrate a partitioning of a clinical environment into eight example regions A-H. Region A includes an ultrasound machine, such as the robot 602 (e.g., the robot 602-A or the robot 602-B). Regions H, B, and C include clinicians 610-1, 610-2, and 610-3, respectively. The robot 602 can move to a location in the environment based on who or what is in a region, instructions received from a clinician, and/or a next step of a procedure as determined by the robot 602. For example, FIG. 6B illustrates an example state machine 650 for autonomous movement of robotic manipulators (e.g., the robots 602-A or 602-B). The state machine 650 includes a first-region state 652, an open-region state 654, a next-step state 656, and a safe-location state 658.

With a current location of the robot 602 being in a first region of the environment (e.g., one of the regions A-H), the state machine 650 starts at the first-region state 652. If the robot 602 determines that no obstructions exist in the first region and if the robot 602 does not determine a next step to perform for a procedure on the patient 116, then the robot 602 remains in the first region and the state machine 650 remains (e.g., arrow 660) in the first-region state 652. If the robot 602 determines there is an obstruction in the first region and another region is open (e.g., unobstructed), the state machine 650 transitions (e.g., arrow 662) to the open-region state 654 and the robot 602 moves to the open region. Subsequently, if the robot 602 determines the first region has become unobstructed, the robot 602 can move back to the first region and the state machine 650 can transition (e.g., arrow 664) to the first-region state 652. Alternatively, the robot 602 can remain at the open region and redefine it as the first region, thus effectively transitioning (e.g., arrow 664) the state machine 650 to the first-region state 652.

From the first-region state 652, the robot 602 can determine a next step of a procedure (e.g., a next protocol step). The robot 602 can then move to a region suitable to perform the next step (e.g., next-step region) and the state machine 650 transitions (e.g., arrow 666) to the next-step state 656. The robot 602 remains in the next-step region and the state machine 650 remains (e.g., arrow 668) in the next-step state 656 while the robot 602 performs the next step. When the next step is completed, the robot 602 can move to a safe location (a predefined location in the environment where the robot 602 is out of the way of the clinicians 610 and the patient 116, such as a corner of a room) and the state machine 650 can transition (e.g., arrow 670) to the safe-location state 658. Further, if the state machine 650 is in the first-region state 652 and the robot 602 determines that all regions are obstructed, the robot 602 can move to the safe location and the state machine 650 can transition (e.g., arrow 672) to the safe-location state 658. The state machine 650 remains (e.g., arrow 674) in the safe-location state 658 while all regions are obstructed. When the robot 602 determines that a region opens or becomes unobstructed, the robot 602 can move to that region and the state machine 650 can transition (e.g., arrow 676) to the open-region state 654. In this way, the robot 602 can autonomously move throughout the environment and provide services to the clinicians 610, all while staying out of the way of the clinicians 610.

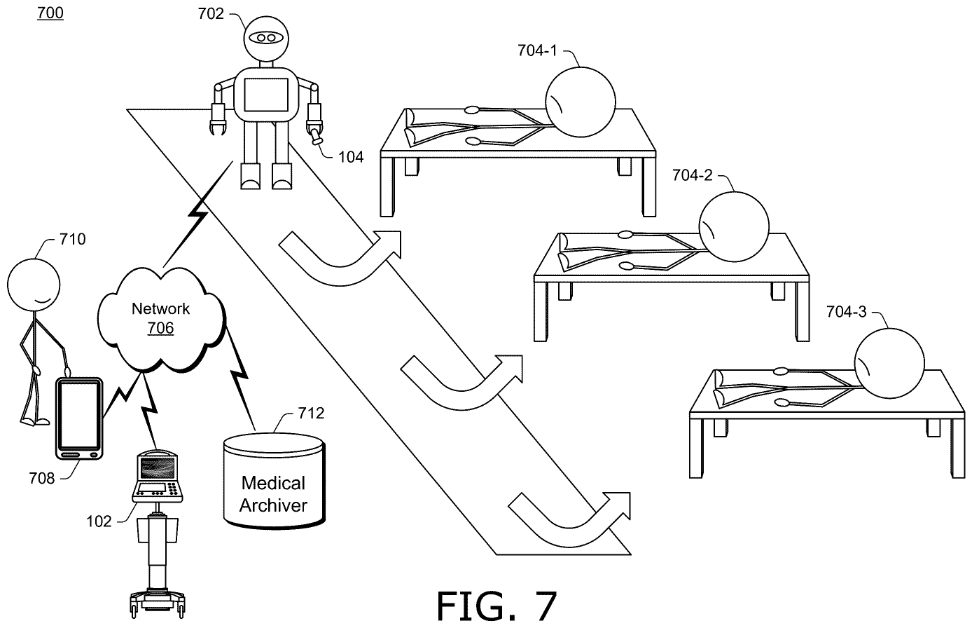

FIG. 7 illustrates an example environment 700 for repeatable ultrasound using multi-array scanners. The environment 700 is an example of an environment for care in a hospital, a care facility, a triage center, a field hospital (e.g., in a war zone), etc. The environment 700 includes a robot 702, which is an example of the robots 602. The environment 700 also includes a row of patients 704-1, 704-2, and 704-3 (collectively patients 704).

The environment 700 also includes a network 706. The robot 702 is communicatively coupled to the network 706, enabling the robot 702 to send information over the network 706 to a device coupled to the network 706 and also enabling the robot 702 to receive information over the network 706 from a device coupled to the network 706. For instance, a computing device 708 (e.g., smart phone, tablet, and the like) is coupled to the network 706 and operated by a clinician 710. In an example, the clinician 710 uses the computing device 708 to provide instructions over the network 706 to the robot 702. The clinician 710 can provide examination instructions to the robot 702 to perform an examination and/or procedure on the patient 704-1. The robot 702 can then determine automatically and without further instruction from the clinician 710 to repeat the examination and/or procedure on one or more additional patients (e.g., the patients 704-2 and 704-3). This repetition can save time and resources in certain types of environments, such as a war zone, a pandemic situation, or a triage center, where staff is necessarily short-handed and patient conditions are unknown. The robot 702 can start with a FAST protocol for each of the patients 704 and, based on what the robot 702 determines during the FAST protocol, can be trained to follow the FAST protocol with another protocol. At least one protocol can use a multi-array scanner. In implementations, the robot 702 performs multiple protocols with a same multi-array scanner (e.g., the ultrasound scanner 104) that remains mounted to the robot 702 for the duration of the multiple protocols for the patients 704.

In an example, the clinician 710 is remote from the environment 700 (e.g., not in the care facility, the examination room, etc.) and observes information from the robot 702 over the network 706 via the computing device 708. The clinician 710 can then, based on the information received, provide instructions to the robot 702, such as to perform a lung examination on one of the patients 704. In an example, the clinician 710 can give a coarse command to the robot 702 and, in response, the robot 702 can implement fine control for the coarse command. For instance, the clinician 710 can issue a coarse command, such as by waving a scanner over lungs of a dummy patient (not shown in FIG. 7) proximate to the clinician 710. In response, the robot 702 can check for a pneumothorax condition on one of the patients 704. In aspects, the robot 702 can learn over time. For instance, the robot 702 can interpret the coarse command from the clinician 710 as the check for the pneumothorax condition based on a pneumothorax condition determined on a previous patient. In this example, the clinician 710 may have previously instructed the robot 702 to check for a pneumothorax condition.

In another example, the robot 702 learns over time based on feedback provided by the clinician 710 to the robot 702 based on the information received from the robot 702. For instance, after the robot 702 performs a first examination on the first patient 704-1, the clinician 710 can instruct the robot 702 to now perform a second examination on the first patient 704-1. The robot 702 can learn to perform the second examination after performing the first examination on a different patient (e.g., the second patient 704-2 and/or the third patient 704-3) if the results of the first examination on the different patient are similar to the results of the first examination of the first patient 704-1.

In some implementations, the clinician 710 and the robot 702 are located in the environment 700 (e.g., an emergency department of a care facility) proximate to the patients 704. When the clinician 710 performs an ultrasound examination on one of the patients 704, the robot 702 can mimic the clinician 710 and perform the same ultrasound examination on one or more of the other patients 704. Additionally or alternatively, the robot 702 can shadow the clinician 710 in the environment 700. For instance, the robot 702 can act as a dependent drone or personal assistant to the clinician 710 to provide any suitable service to the clinician 710 as the clinician 710 works on the patients 704. Additionally or alternatively, the robot 702 can learn from the clinician 710 as the robot 702 shadows the clinician 710 in the environment 700. In this way, the robot 702 is an adaptive system that can adapt by learning from the clinician 710.

In some implementations, the clinician 710 can control the robot 702 remotely. For instance, the clinician 710 can wear augmented-reality/virtual-reality (AR/VR) goggles that display one of the patients 704 and an ultrasound image generated by the robot 702 (e.g., by the ultrasound scanner 104 held by or mounted to the robot 702).

Moreover, the environment 700 includes an ultrasound machine (e.g., the ultrasound machine 102) and a medical archiver 712 communicatively coupled to the network 706. Hence, the robot 702 can provide ultrasound data, generated by the ultrasound scanner 104 held by or mounted to the robot 702, to the ultrasound machine 102. Then, the ultrasound machine 102 can generate ultrasound images from the ultrasound data. In some implementations, the robot 702 includes the ultrasound machine 102. Ultrasound data generated by the ultrasound machine 102 and/or the robot 702 (e.g., by the ultrasound scanner 104 held by or mounted to the robot 702) can be sent via the network 706 to the medical archiver 712. Further, the medical archiver 712 can provide data over the network 706. Hence, the computing device 708, the ultrasound machine 102, and/or the robot 702 can compare ultrasound data from a previous ultrasound examination to the ultrasound data from the current ultrasound examination.

### Example Machine-Learned Model

Many of the aspects described herein can be implemented using a machine-learned model. For the purposes of this disclosure, a machine-learned model is any model that accepts an input, analyzes and/or processes the input based on an algorithm derived via machine-learning training, and provides an output. A machine-learned model can be conceptualized as a mathematical function of the following form:

$$f ( \hat{s} , θ ) = \hat{y} \qquad Equation ( 1 )$$

In Equation (1), the operator f represents the processing of the machine-learned model based on an input and providing an output. The term ŝ represents a model input, such as ultrasound data. The model analyzes/processes the input ŝ using parameters θ to generate output ŷ (e.g., object identification, object segmentation, object classification, etc.). Both ŝ and ŷ can be scalar values, matrices, vectors, or mathematical representations of phenomena such as categories, classifications, image characteristics, the images themselves, text, labels, or the like. The parameters θ can be any suitable mathematical operations, including but not limited to applications of weights and biases, filter coefficients, summations or other aggregations of data inputs, distribution parameters such as mean and variance in a Gaussian distribution, linear algebra-based operators, or other parameters, including combinations of different parameters, suitable to map data to a desired output.

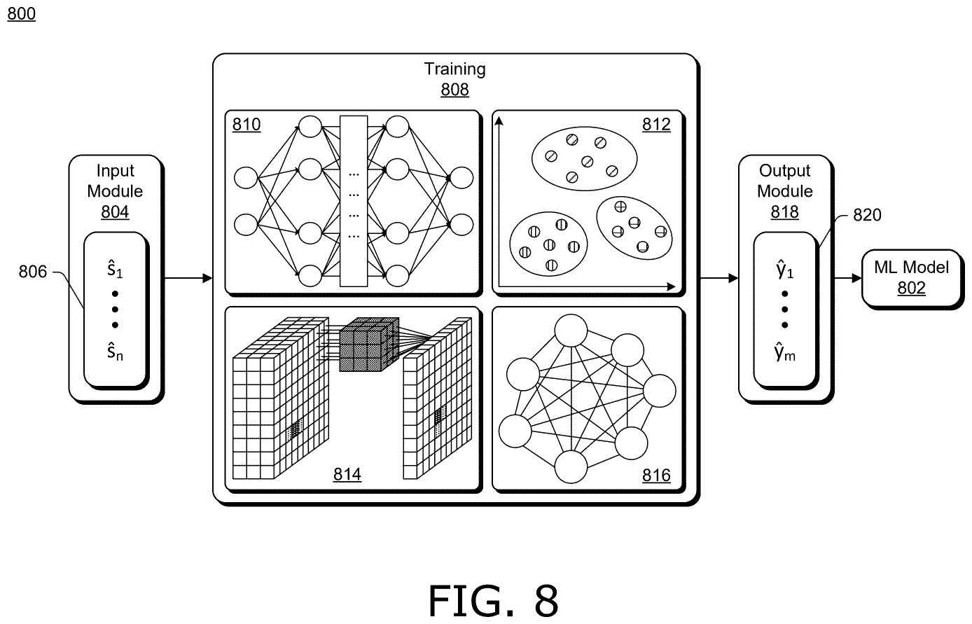

FIG. 8 represents an example machine-learning architecture 800 used to train a machine-learned model M 802. An input module 804 accepts an input ŝ 806, which can be an array with members ŝ₁ through ŝₙ. The input ŝ 806 is fed into a training module 808, which processes the input ŝ 806 based on the machine-learning architecture 800. For example, if the machine-learning architecture 800 uses a multilayer perceptron (MLP) model 810, the training module 808 applies weights and biases to the input ŝ 806 through one or more layers of perceptrons, each perceptron performing a fit using its own weights and biases according to its given functional form. MLP weights and biases can be adjusted so that they are optimized against a least mean square, logcosh, or other optimization function (e.g., loss function) known in the art. Although an MLP model 810 is described here as an example, any suitable machine-learning technique can be employed, some examples of which include but are not limited to k-means clustering 812, convolutional neural networks (CNN) 814, a Boltzmann machine 816, Gaussian mixture models (GMM), and long short-term memory (LSTM). The training module 808 provides an input to an output module 818. The output module 818 analyzes the input from the training module 808 and provides an output in the form of ŷ 820, which can be an array with members ŷ₁ through ŷₘ. The output 820 can represent a known correlation with the input ŝ 806, such as, for example, object identification, segmentation, and/or classification.

In some examples, the input ŝ 806 can be a training input labeled with known output correlation values, and these known values can be used to optimize the output ŷ 820 in training against the optimization/loss function. In other examples, the machine-learning architecture 800 can categorize the output ŷ 820 values without being given known correlation values to the inputs ŝ 806. In some examples, the machine-learning architecture 800 can be a combination of machine-learning architectures. By way of example, a first network can use the input ŝ 806 and provide the output ŷ 820 as an input SML to a second machine-learned architecture, with the second machine-learned architecture providing a final output ŷ_{f}. In another example, one or more machine-learning architectures can be implemented at various points throughout the training module 808.

In some machine-learned models, all layers of the model are fully connected. For example, all perceptrons in an MLP model act on every member of ŝ. For an MLP model with a 100×100 pixel image as the input, each perceptron provides weights/biases for 10,000 inputs. With a large, densely layered model, this may result in slower processing and/or issues with vanishing and/or exploding gradients. A CNN, which may not be a fully connected model, can process the same image using 5×5 tiled regions, requiring only 25 perceptrons with shared weights, giving much greater efficiency than the fully connected MLP model.

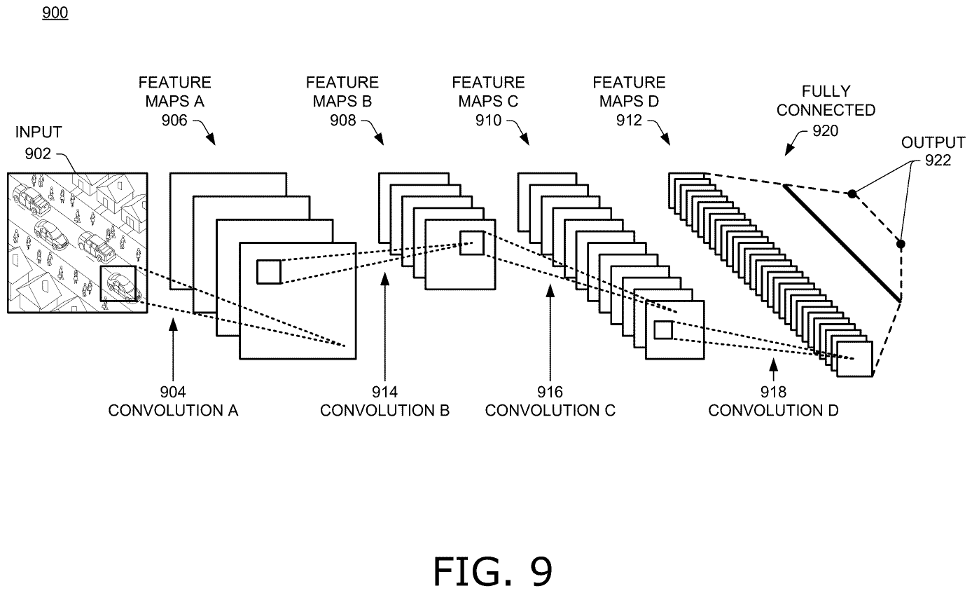

FIG. 9 represents an example model 900 using a CNN to process an input image 902, which includes representations of objects that can be identified via object recognition, such as people or cars (or an anatomy, as described in relation to FIGS. 1-8). Convolution A 904 can be performed to create a first set of feature maps (e.g., feature maps A 906). A feature map can be a mapping of aspects of the input image 902 given by a filter element of the CNN. This process can be repeated using the feature maps A 906 to generate further feature maps B 908, feature maps C 910, and feature maps D 912 using convolution B 914, convolution C 916, and convolution D 918, respectively. In this example, the feature maps D 912 become an input for fully connected network layers 920. In this way, the machine-learned model can be trained to recognize certain elements of the image 902, such as people, cars, or a particular patient anatomy, and provide an output 922 that, for example, identifies the recognized elements. In some aspects, an inference generated with an ultrasound system can be appended to a feature vector, such as a feature map (e.g., feature map B 908), generated by a neural network (e.g., CNN). In this way, the feature vector and/or inference can be used as a secondary/conditional input to the neural network.

Although the example of FIG. 9 shows a CNN as a part of a fully connected network, other architectures are possible and this example should not be seen as limiting. There can be more or fewer layers in the CNN. A CNN component for a model can be placed in a different order, or the model can contain additional components or models. There may be no fully connected components, such as a fully convolutional network. Additional aspects of the CNN, such as pooling, downsampling, upsampling, or other aspects known to people skilled in the art, can also be employed.

### Example Device

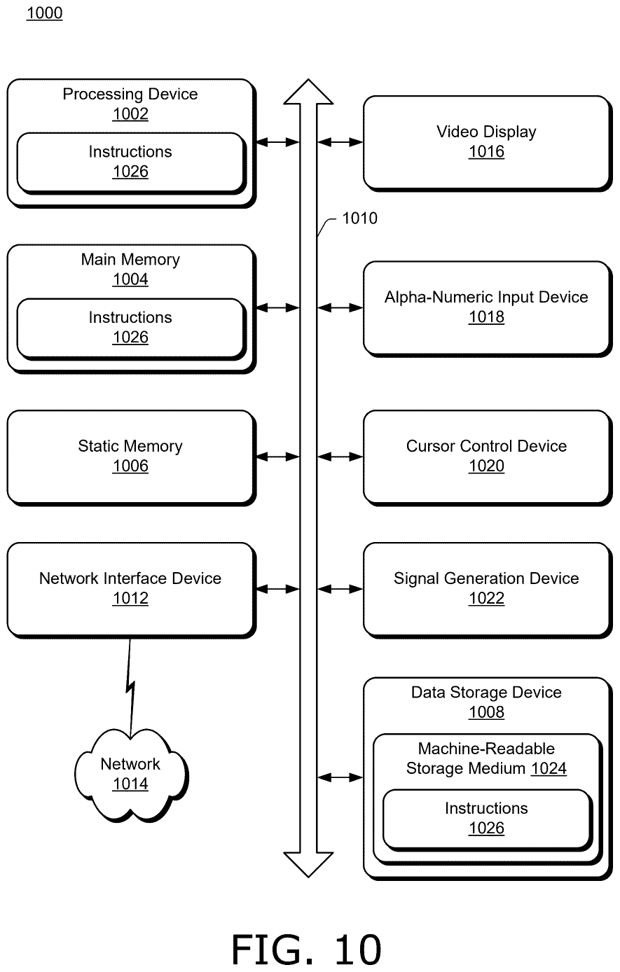

FIG. 10 illustrates a block diagram of an example computing device 1000 that can perform one or more of the operations described herein, in accordance with some implementations. The computing device 1000 can be connected to other computing devices in a local area network (LAN), an intranet, an extranet, and/or the Internet. The computing device 1000 can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, an ultrasound machine, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some implementations, the computing device 1000 is one or more of an ultrasound machine, an ultrasound scanner, an access point, a charging station, and a medical archiver.

The example computing device 1000 can include a processing device 1002 (e.g., a general-purpose processor, a programmable logic device (PLD), etc.), a main memory 1004 (e.g., synchronous dynamic random-access memory (DRAM), read-only memory (ROM), etc.), and a static memory 1006 (e.g., flash memory, a data storage device 1008, etc.), which can communicate with each other via a bus 1010. The processing device 1002 can be provided by one or more general-purpose processing devices such as a microprocessor, a central processing unit, or the like. In an illustrative example, the processing device 1002 comprises a CISC microprocessor, a RISC microprocessor, a VLIW microprocessor, or a processor implementing other instruction sets or processors implementing a combination of instruction sets. The processing device 1002 can also comprise one or more special-purpose processing devices such as an application-specific integrated circuit (ASIC), a field programmable gate array (FPGA), a digital signal processor (DSP), a network processor, and the like. The processing device 1002 can be configured to execute the operations described herein, in accordance with one or more aspects of the present disclosure.

The computing device 1000 can further include a network interface device 1012, which can communicate with a network 1014. The computing device 1000 also can include a video display unit 1016 (e.g., a liquid crystal display (LCD), an organic light-emitting diode (OLED), a cathode ray tube (CRT), etc.), an alphanumeric input device 1018 (e.g., a keyboard), a cursor control device 1020 (e.g., a mouse), and an acoustic signal generation device 1022 (e.g., a speaker, a microphone, etc.). In one implementation, the video display unit 1016, the alphanumeric input device 1018, and the cursor control device 1020 can be combined into a single component or device (e.g., an LCD touch screen).

The data storage device 1008 can include a computer-readable storage medium 1024 on which can be stored one or more sets of instructions 1026 (e.g., instructions for carrying out the operations described herein, in accordance with one or more aspects of the present disclosure). The instructions 1026 can also reside, completely or at least partially, within the main memory 1004 and/or within the processing device 1002 during execution thereof by the computing device 1000, where the main memory 1004 and the processing device 1002 also constitute computer-readable media. The instructions can further be transmitted or received over the network 1014 via the network interface device 1012.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein are embodied in the data storage device 1008 of the computing device 1000 as executable instructions or code. Although represented as software implementations, the modules described can be implemented as any form of a control application, a software application, a signal processing and control module, hardware, or firmware installed on the computing device 1000.

While the computer-readable storage medium 1024 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### Example Methods

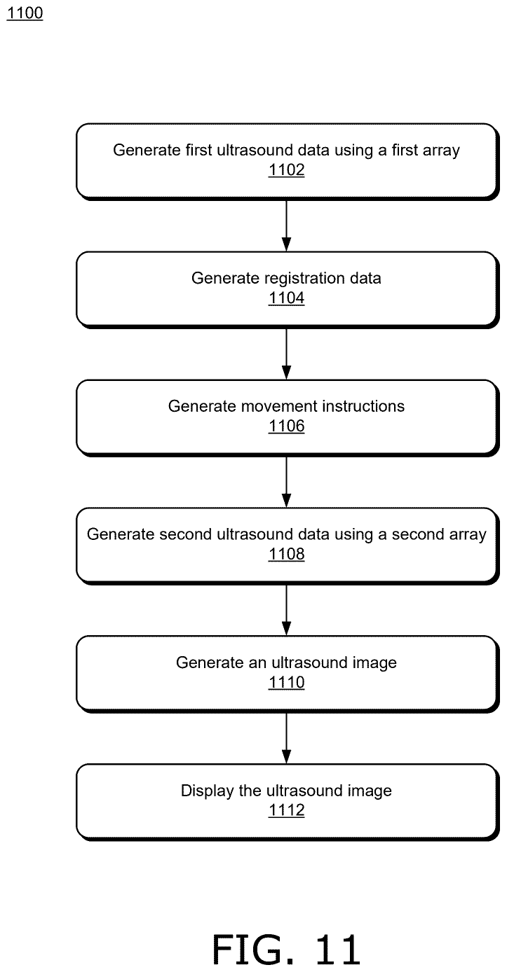

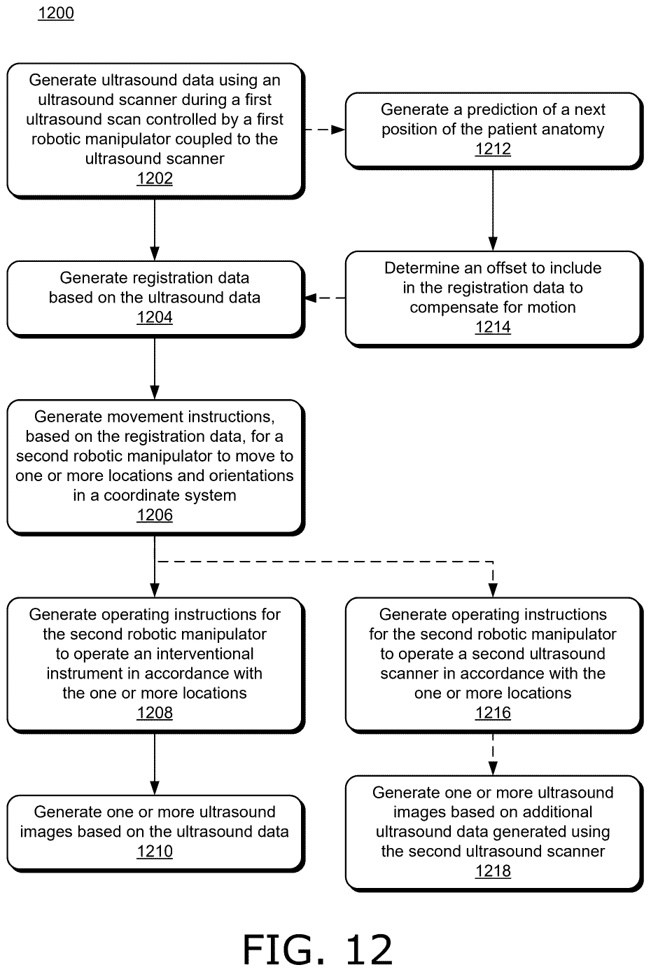

FIGS. 11 and 12 depict methods 1100 and 1200, respectively, for repeatable ultrasound using multi-array scanners. The methods 1100 and 1200 are shown as a set of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example system 100 of FIG. 1 or to entities or processes as detailed in FIGS. 2-10, reference to which is made for example only. The techniques are not limited to performance by one entity or multiple entities operating on one device.

FIG. 11 depicts a method 1100 for repeatable ultrasound using multi-array scanners, in accordance with one or more implementations. The method 1100 can be performed by the ultrasound system 100. For example, the ultrasound system 100 can use one or more robotic manipulators 114, which can couple to one or more scanners 104, such as the ultrasound scanners 132, to implement the method 1100 in accordance with techniques disclosed herein.

At 1102, first ultrasound data is generated based on received reflections of first ultrasound signals transmitted by a first array of a multi-array ultrasound scanner at a patient anatomy. The first ultrasound data can be generated by the scanner 104 of the ultrasound machine 102. In one example, the multi-array scanner 504 is used to generate the ultrasound data 512 based on ultrasound signals transmitted by the multi-array scanner 504 via the first array 506 or the second array 508.

At 1104, registration data is generated based on the first ultrasound data. For example, the registration data 430 is generated by the processor system 414 based on the ultrasound data 512. The processor system 414 sends the registration data 430 to the position controller 416. In some instances, the registration data 430 is generated based on a combination of the ultrasound data 512, the perspective data 426, and the secondary data 428. In some aspects, the registration data is adjusted based on the perspective data to compensate for motion of the patient anatomy, where the motion of the patient anatomy is represented by the amount of movement in the perspective data.

At 1106, movement instructions are generated for a robotic manipulator coupled to the multi-array ultrasound scanner based on the registration data. In aspects, the movement instructions are configured to cause the robotic manipulator to move to one or more locations in a coordinate system. In an example, the position controller 416 generates the movement instructions 510 to instruct the robotic manipulator 502 to move to one or more locations and orientations in a coordinate system, such as the coordinate system 126, to enable the second array 508 to be used to generate ultrasound data in a desired image plane and with a desired view/perspective. In another example, the movement instructions 510 are generated by the position controller 416 to instruct the second robotic manipulator 404 to move to one or more locations and orientations in the coordinate system 126 to enable the second robotic manipulator 404 to operate an interventional instrument, such as the needle 410. In one example, the movement instructions 510 include instructions for the robotic manipulator 502 to move to one or more locations and orientations corresponding to another patient to enable the second array 508 to be used to generate additional ultrasound data from the other patient. In some implementations, the robotic manipulator is a single robotic manipulator. In some implementations, a position controller can be configured to receive pre-tune data usable to generate initial movement instructions for the one or more robotic manipulators, where the initial movement instructions are configured to cause the one or more robotic manipulators to begin an ultrasound scan without explicit positional instructions provided by a clinician or derived from a current use of the ultrasound system.

In some aspects, the movement instructions can compensate for motion of the patient anatomy to enable the one or more robotic manipulators to dynamically adjust for the motion. The motion can be determined based on positional differences of the patient anatomy over a set of ultrasound images generated from the ultrasound data using the first array over a duration of time, a prediction of a next position of the patient anatomy relative to a current position of the patient anatomy, wherein the prediction is generated based on the positional differences of the patient anatomy determined from the set of ultrasound images, and an offset representing the prediction, where the offset is included in the registration data to enable the movement instructions generated from the registration data to cause the one or more robotic manipulators to dynamically adjust for the motion when operating the one or more scanners to generate the additional ultrasound data using the second array.

At 1108, second ultrasound data is generated based on received reflections of second ultrasound signals transmitted by a second array of the multi-array ultrasound scanner at the patient anatomy in accordance with the one or more locations. In one example, the robotic manipulator 502, coupled to the multi-array scanner 504, uses the movement instructions 510 to move the multi-array scanner 504 such that the multi-array scanner 504 can generate ultrasound data using the second array 508 in accordance with the operating instructions 432 (e.g., scan instructions). In some implementations, the operating instructions 432 configure the second array 508 for an imaging mode (B-mode, super harmonic tissue imaging, sub-harmonic tissue imaging, etc.)

At 1110, an ultrasound image is generated based on the first ultrasound data, the second ultrasound data, or both the first ultrasound data and the second ultrasound data. In one example, the image generator 418 generates one or more ultrasound images 424 (e.g., the ultrasound image 118, the ultrasound image 606) based on the ultrasound data 422 or the ultrasound data 512. In one implementation, the ultrasound image 424 is based on the ultrasound data 422 generated by the scanner 406 or based on the ultrasound data 512 generated by the multi-array scanner 504 using the first array 506. In another example, the ultrasound image 424 is based on ultrasound data generated by the scanner 408 or based on the ultrasound data 512 generated by the multi-array scanner 504 using the second array 508. In yet another example, the ultrasound image 424 is generated using the ultrasound data 422 generated by the scanner 406 in combination with ultrasound data generated by the scanner 408. In another implementation, the ultrasound image 424 is generated by the multi-array scanner 504 based on a combination of ultrasound data generated using the first array 506 and ultrasound data generated using the second array 508. In some aspects, the ultrasound image is a 3D image of the patient anatomy, which is generated based on the first ultrasound data and the second ultrasound data being generated at different imaging planes.

At 1112, the ultrasound image is displayed via a display device. In an example, the ultrasound image 424 (e.g., the ultrasound image 118) is displayed via the display device 108 of the ultrasound machine 102. In another example, the ultrasound image 606 is displayed via the display device 604 on a robot (e.g., the first robot 602-A, the second robot 602-B). In some implementations, the ultrasound system can receive examination instructions from a user for performing an ultrasound examination on a first patient having the patient anatomy and determine automatically and without further instructions from the user to repeat the ultrasound examination on one or more additional patients.

FIG. 12 depicts a method 1200 for repeatable ultrasound using multi-array scanners, in accordance with one or more implementations. The method 1200 can be performed by the ultrasound system 100. For example, the ultrasound system 100 can use one or more robotic manipulators 114, which can couple to one or more scanners 104, such as the ultrasound scanners 132, to implement the method 1200 in accordance with techniques disclosed herein.

At 1202, ultrasound data is generated using an ultrasound scanner during a first ultrasound scan. The ultrasound data can be generated by the scanner 104 of the ultrasound machine 102. In one example, the scanner 406 is used to generate the ultrasound data 422 based on ultrasound signals transmitted by the scanner 406 at the anatomy of the patient 116. In another example, the multi-array scanner 504 is used to generate the ultrasound data 512 based on ultrasound signals transmitted by the multi-array scanner 504 via the first array 506 or the second array 508. In some implementations, a position controller can be configured to receive pre-tune data usable to generate initial movement instructions for the one or more robotic manipulators, the initial movement instructions configured to cause the one or more robotic manipulators to begin an ultrasound scan (e.g., the first ultrasound scan) without explicit positional instructions provided by a clinician or derived from a current use of the ultrasound system.

At 1204, registration data is generated based on the ultrasound data. For example, the registration data 430 is generated by the processor system 414 based on the ultrasound data 422 or the ultrasound data 512. In some examples, the registration data 430 is generated based on a combination of the ultrasound data 422 or 512, perspective data 426 received from a machine-learned model (e.g., the machine-learned model 420), and secondary data (e.g., the secondary data 428) corresponding to the patient anatomy. The perspective data 426 represents a perspective for an ultrasound image generated from the ultrasound data. The secondary data can include one or more of physiological data, previous scan data or images of the patient anatomy, inertial measurement unit data, protocol data, and clinician instructions.

At 1206, movement instructions are generated, based on the registration data, for one or more robotic manipulators to move to one or more locations and orientations in a coordinate system. In an example, the position controller 416 generates the movement instructions 434 to instruct the second robotic manipulator 404 to move to one or more locations and orientations in the coordinate system 126, to enable the scanner 408 to generate ultrasound data in a desired image plane and with a desired view/perspective. In another example, the movement instructions 434 are generated by the position controller 416 to instruct the second robotic manipulator 404 to move to one or more locations and orientations in the coordinate system 126 to enable the second robotic manipulator 404 to operate an interventional instrument, such as the needle 410. In another example, the movement instructions 434 include instructions for the second robotic manipulator 404 to perform a second ultrasound scan on one or more additional patients, such that the second ultrasound scan is a repeat of the first ultrasound scan on the one or more additional patients. In one example, the second robotic manipulator 404 and the first robotic manipulator 402 are coupled to the same robot. In another example, the first and second robotic manipulators are separate robots.

In some implementations, the first robotic manipulator is configured to couple to and control first positioning, first movement, and first operation of the first ultrasound scanner, the second robotic manipulator is configured to couple to and control second positioning, second movement, and second operation of the second ultrasound scanner, the first ultrasound scanner is configured to generate the ultrasound data during the first ultrasound scan, and the second ultrasound scanner is configured to generate the additional ultrasound data during the second ultrasound scan. In some aspects, the movement instructions are configured to cause the second robotic manipulator to move to the one or more locations in the coordinate system, and the scan instructions configure the second ultrasound scanner for the imaging mode.

In some aspects, the movement instructions can compensate for motion of the patient anatomy to enable the one or more robotic manipulators to dynamically adjust for the motion. The motion can be determined based on positional differences of the patient anatomy over a set of ultrasound images generated from the ultrasound data using the first array over a duration of time, a prediction of a next position of the patient anatomy relative to a current position of the patient anatomy, wherein the prediction is generated based on the positional differences of the patient anatomy determined from the set of ultrasound images, and an offset representing the prediction, where the offset is included in the registration data to enable the movement instructions generated from the registration data to cause the one or more robotic manipulators to dynamically adjust for the motion when operating the one or more scanners to generate the additional ultrasound data using the second array.

At 1208, operating instructions are generated for the second robotic manipulator to operate an interventional instrument in accordance with the one or more locations. The operating instructions can include instructions for inserting the interventional instrument into the patient anatomy and injecting or extracting fluid or tissue via the interventional instrument. For example, the processor system 414 can generate the operating instructions 432 to cause the second robotic manipulator 404 to use the needle 410 to inject fluid into the patient anatomy or to extract fluid from the patient anatomy. The operating instructions 432 are used in combination with the movement instructions 434 for inserting and removing the needle 410 from the patient at a desired location and with a desired orientation.

At 1210, additional ultrasound data is generated using the ultrasound scanner during the second ultrasound scan. For example, the scanner 408 generates additional ultrasound data of the patient anatomy or of an anatomy of another patient, based on the operating instructions 432 and in accordance with movement controlled by the second robotic manipulator 404 as instructed by the movement instructions 434.

In some examples, at 1212, a prediction of a next position of the patient anatomy is generated. The prediction of the next position is relative to a current position of the patent anatomy. In an example, the motion-compensation system 438 uses a set of ultrasound images 424 generated over a duration of time, such as one or more breathing cycles of the patient, to determine the patient's motion and predict a next position of the patient in accordance with the determined motion. For example, the motion-compensation system 438 determines positional differences of the patient anatomy over the set of ultrasound images.

Following 1212, at 1214, an offset to include in the registration data to dynamically compensate for motion of the patient anatomy is determined. The offset is calculated based on the predicted next position of the patient and included in the registration data 430. In an example, the offset included in the registration data 430 is used by the position controller 416 when generating the movement instructions 434 for the second robotic manipulator 404. The offset compensates for motion of the patient, enables consistent and stable ultrasound images that reduce artifacts caused by motion to be generated, and enhances the accuracy of needle insertion into the patient. The method 1200 can then return to 1204 to generate the registration data with the offset to compensate for motion of the patient.

In some examples, following 1206, at 1216, operating instructions are generated for the second robotic manipulator to operate a second ultrasound scanner in accordance with the one or more locations. For example, the processor system 414 generates operating instructions 432 (e.g., scan instructions) to configure the scanner 408 or the multi-array scanner 504 for an imaging mode for a second ultrasound scan. The second ultrasound scan can include using the scanner 408 in an imaging mode that is different from an imaging mode used by the scanner 406. In another example, the second ultrasound scan can include using the scanner 408 in an imaging plane that is different from an imaging plane used by the scanner 406. In another example, the second ultrasound scan can include using the second array 508 of the multi-array scanner 504 in a different imaging mode than the first array 506. In one example, the ultrasound system can receive examination instructions from a user for performing an ultrasound examination (e.g., operations 1202 to 1210, operations 1202 to 1214, or operations 1202 to 1218) on a first patient having the patient anatomy and then determine automatically and without further instructions from the user to repeat the ultrasound examination on one or more additional patients. In some aspects, the ultrasound examination includes the first ultrasound scan and the second ultrasound scan.

Following 1216, at 1218, one or more ultrasound images are generated based on additional ultrasound data generated using the second ultrasound scanner. For example, the scanner 408 generates additional ultrasound data in accordance with the operating instructions 432 and the positioning as controlled by the second robotic manipulator 404. This additional ultrasound data is used by the ultrasound system 400 to generate ultrasound images, such as the ultrasound image 118 and ultrasound images 606.

While the present subject matter has been described in detail with respect to various specific example implementations thereof, each example is provided by way of explanation and not limitation of the disclosure. Those skilled in the art, upon attaining an understanding of the foregoing, can readily produce alterations to, variations of, and equivalents to such implementations. Accordingly, the subject disclosure does not preclude inclusion of such modifications, variations, and/or additions to the present subject matter as would be readily apparent to one of ordinary skill in the art. For instance, features illustrated or described as part of one implementation can be used with another implementation to yield a still further implementation. Thus, it is intended that the present disclosure cover such alterations, variations, and equivalents.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (a-a, a-a-a, a-a-b, a-a-c, a-b-b, a-c-c, b-b, b-b-b, b-b-c, c-c, and c-c-c or any other ordering of a, b, and c).

While various implementations of the disclosure are described in the foregoing description and shown in the drawings, it is to be distinctly understood that this disclosure is not limited thereto but may be variously embodied to practice within the scope of the following claims. From the foregoing description, it will be apparent that various changes may be made without departing from the spirit and scope of the disclosure as defined by the following claims.

### CONCLUSION

Implementations for repeatable ultrasound using multi-array scanners are disclosed. The techniques disclosed herein provide solutions that enable consistent and stable ultrasound images to be generated on one or more patients, resulting in enhanced patient care. These solutions reduce artifacts caused by motion or caused by switching ultrasound scanners. These techniques further enable ultrasound examinations to be repeated on multiple patients or on the same patient at different times.

What is claimed is:

1. An ultrasound system comprising:
one or more ultrasound scanners configured to generate ultrasound data based on received reflections of ultrasound signals transmitted by the one or more ultrasound scanners at a patient anatomy during a first ultrasound scan;
one or more robotic manipulators configured to couple to the one or more ultrasound scanners, the one or more robotic manipulators configured to control positioning, movement, and operation of the one or more ultrasound scanners in accordance with a coordinate system;
a processor system configured to:
receive the ultrasound data from the one or more ultrasound scanners;
generate registration data based on the ultrasound data; and
generate, based on the ultrasound data, scan instructions to configure the one or more ultrasound scanners for an imaging mode for a second ultrasound scan; and
a position controller configured to generate movement instructions for the one or more robotic manipulators based on the registration data, the movement instructions configured to cause the one or more robotic manipulators to move to one or more locations in the coordinate system to enable the one or more ultrasound scanners to perform the second ultrasound scan at the one or more locations in accordance with the imaging mode to generate additional ultrasound data.

2. The ultrasound system of claim 1, wherein:
the one or more robotic manipulators include a single robotic manipulator;
the one or more ultrasound scanners include a multi-array ultrasound scanner having a first array and a second array;
the ultrasound data is generated using the first array; and
the additional ultrasound data is generated using the second array.

3. The ultrasound system of claim 2, wherein:
the movement instructions are configured to cause the one or more robotic manipulators to move to the one or more locations in the coordinate system; and
the scan instructions configure the second array for the imaging mode.

4. The ultrasound system of claim 2, further comprising a motion-compensation system configured to compensate for motion of the patient anatomy to enable the one or more robotic manipulators to dynamically adjust for the motion, the motion determined based on:
positional differences of the patient anatomy over a set of ultrasound images generated from the ultrasound data using the first array over a duration of time;
a prediction of a next position of the patient anatomy relative to a current position of the patient anatomy, wherein the prediction is generated based on the positional differences of the patient anatomy determined from the set of ultrasound images; and
an offset representing the prediction, wherein the offset is included in the registration data to enable the movement instructions generated from the registration data to cause the one or more robotic manipulators to dynamically adjust for the motion when operating the one or more scanners to generate the additional ultrasound data using the second array.

5. The ultrasound system of claim 1, wherein the position controller is configured to receive pre-tune data usable to generate initial movement instructions for the one or more robotic manipulators, the initial movement instructions configured to cause the one or more robotic manipulators to begin an ultrasound scan without explicit positional instructions provided by a clinician or derived from a current use of the ultrasound system.

6. The ultrasound system of claim 1, wherein:
the processor system is further configured to:
receive examination instructions from a user for performing an ultrasound examination on a first patient having the patient anatomy, the ultrasound examination including the first ultrasound scan and the second ultrasound scan; and
determine automatically and without further instructions from the user to repeat the ultrasound examination on one or more additional patients.

7. The ultrasound system of claim 1, wherein:
the one or more robotic manipulators include a first robotic manipulator and a second robotic manipulator;
the one or more ultrasound scanners include a first ultrasound scanner and a second ultrasound scanner;
the first robotic manipulator is configured to couple to and control first positioning, first movement, and first operation of the first ultrasound scanner;
the second robotic manipulator is configured to couple to and control second positioning, second movement, and second operation of the second ultrasound scanner;
the first ultrasound scanner is configured to generate the ultrasound data during the first ultrasound scan; and
the second ultrasound scanner is configured to generate the additional ultrasound data during the second ultrasound scan.

8. The ultrasound system of claim 7, wherein:
the movement instructions are configured to cause the second robotic manipulator to move to the one or more locations in the coordinate system; and
the scan instructions configure the second ultrasound scanner for the imaging mode.

9. The ultrasound system of claim 7, wherein the first robotic manipulator and the second robotic manipulator are coupled to a same robot.

10. The ultrasound system of claim 7, wherein the first robotic manipulator and the second robotic manipulator are separate robots.

11. An ultrasound system comprising:
a multi-array ultrasound scanner having at least a first array and a second array, the multi-array ultrasound scanner configured to generate ultrasound data based on received reflections of ultrasound signals transmitted by the multi-array ultrasound scanner at a patient anatomy during a first ultrasound scan, the ultrasound data including first ultrasound data generated using the first array and second ultrasound data generated using the second array;
a first robotic manipulator configured to couple to the multi-array ultrasound scanner, the first robotic manipulator configured to control first positioning, first movement, and first operation of the multi-array ultrasound scanner in accordance with a coordinate system;
a second robotic manipulator configured to couple to an interventional instrument, the second robotic manipulator configured to control second positioning, second movement, and second operation of the interventional instrument in accordance with the coordinate system;
a processor system configured to:
receive the ultrasound data from the multi-array ultrasound scanner;
generate registration data based on the ultrasound data; and
generate operating instructions for the second robotic manipulator to operate the interventional instrument based on the ultrasound data; and
a position controller configured to generate movement instructions for the second robotic manipulator based on the registration data, the movement instructions configured to cause the second robotic manipulator to move to one or more locations and orientations in the coordinate system to enable the operation of the interventional instrument at the one or more locations and orientations in accordance with the operating instructions.

12. The ultrasound system of claim 11, wherein:
the operating instructions include instructions for inserting the interventional instrument into the patient anatomy and injecting or extracting fluid or tissue via the interventional instrument.

13. The ultrasound system of claim 11, further comprising a motion-compensation system configured to:
generate, based on a set of ultrasound images generated from the ultrasound data, a prediction of a next position of the patient anatomy relative to a current position of the patient anatomy; and
determine, based on the prediction, an offset to include in the registration data to adjust the movement instructions generated for the second robotic manipulator to dynamically compensate for motion of the patient anatomy.

14. The ultrasound system of claim 11, wherein the processor system is configured to:
receive perspective data from a machine-learned model, the perspective data representing a perspective for an ultrasound image generated from the ultrasound data;
receive secondary data corresponding to the patient anatomy; and
generate the registration data based on a combination of the ultrasound data, the perspective data, and the secondary data.

15. The ultrasound system of claim 14, wherein the secondary data includes one or more of physiological data, previous scan data or images of the patient anatomy, inertial measurement unit data, protocol data, and clinician instructions.

16. A method for repeatable ultrasound using multi-array scanners, the method comprising:
generating first ultrasound data based on first received reflections of first ultrasound signals transmitted by a first array of a multi-array ultrasound scanner at a patient anatomy;
generating registration data based on the first ultrasound data;
generating movement instructions for a robotic manipulator coupled to the multi-array ultrasound scanner based on the registration data, the movement instructions configured to cause the robotic manipulator to move to one or more locations in a coordinate system; and
generating second ultrasound data based on second received reflections of second ultrasound signals transmitted by a second array of the multi-array ultrasound scanner at the patient anatomy in accordance with the one or more locations.

17. The method of claim 16, further comprising:
generating an ultrasound image based on the first ultrasound data, the second ultrasound data, or both the first ultrasound data and the second ultrasound data; and
displaying the ultrasound image via a display device.

18. The method of claim 16, wherein:
the first ultrasound data and the second ultrasound data are generated at different imaging planes from one another; and
the method further comprises generating a three-dimensional image of the patient anatomy based on the first ultrasound data from the first array and the second ultrasound data from the second array.

19. The method of claim 16, further comprising:
generating ultrasound images based on the first ultrasound data;
receiving perspective data from a machine-learned model, the perspective data representing a perspective for each of the ultrasound images and including an amount of movement of the perspective from one of the ultrasound images to another one of the ultrasound images; and
adjusting the registration data based on the perspective data to compensate for motion of the patient anatomy, the motion of the patient anatomy represented by the amount of movement in the perspective data.

20. The method of claim 16, wherein:
generating the first ultrasound data, generating the registration data, generating the movement instructions, and generating the second ultrasound data are operations included in an ultrasound examination; and
the method further comprises:
receiving examination instructions from a user for performing the ultrasound examination on a first patient having the patient anatomy; and
determining automatically and without further instructions from the user to repeat the ultrasound examination on one or more additional patients.
